(19) United States
(12) Patent Application Publication
(10) Pub. No.: US 2024/0389949 A1
(43) Pub. Date: Nov. 28, 2024

(54) SYSTEMS, METHODS, AND APPARATUSES FOR IMPLEMENTING CLOUD-BASED HEALTH, NUTRITIONAL, AND BODY COMPOSITION ANALYSIS

(71) Applicant: Arizona Board of Regents on behalf of Arizona State University, Scottsdale, AZ (US)

(72) Inventors: Paolo De Cristofaro, Giulianova (IT); Andrea De Cristofaro, Roseto degli Abruzzi (IT); Marco Santello, Gilbert, AZ (US); Cristian Currò, Reggio Calabria (IT); Giuseppe Mallamaci, Agrate Brianza (IT); Fabio De Cristofaro, Giulianova (IT)

(21) Appl. No.: 18/694,922

(22) PCT Filed: Oct. 20, 2022
(86) PCT No.: PCT/US2022/047261

§ 371 Date: Mar. 22, 2024

Related U.S. Application Data
(60) Provisional application No. 63/257,902, filed on Oct. 20, 2021.

(51) Int. Cl. A61B 5/00; A61B 5/107; G16H 10/60; G16H 15/00

(52) U.S. Cl. CPC A61B 5/7275; A61B 5/1075; A61B 5/743; A61B 5/7435; G16H 10/60; G16H 15/00

(57) ABSTRACT

Described herein are means for implementing a cloud-based health, nutritional, and body composition analysis platform, in which models are utilized to provide personalized data for health and nutritional counseling. For example, there is a there is a specially configured system (e.g., a “MORPHOGRAM” cloud-based platform) having means for prompting transmitting a GUI for display via a user device specifying instructions to manually determine and enter body metric measurements for a patient; means for receiving inputs providing one or more of (i) the body metric for the patient, (ii) pedometer data for the patient, and (iii) fitness data for the patient; means for populating a personalized risk monitoring profile for the patient by calculating anthropometric indicators; determining a physical activity level for the patient; determining a body type; calculating a physiological lean body mass and percentage of body fat; calculating a target physiological; and re-transmitting the GUI to display the personalized risk monitoring profile for the patient based on the inputs received, determined. Other related embodiments are disclosed.

### CLAIM OF PRIORITY

This International Utility PCT patent application claims priority to the U.S. Provisional patent application No. 63/257,902 (Attorney Docket No. 37684.670P), filed Oct. 20, 2021, entitled “SYSTEMS, METHODS, AND APPARATUSES FOR IMPLEMENTING CLOUD-BASED HEALTH, NUTRITIONAL, AND BODY COMPOSITION ANALYSIS,” the entire contents of which are incorporated herein by reference as though set forth in full.

### GOVERNMENT RIGHTS AND GOVERNMENT AGENCY SUPPORT NOTICE

None.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate generally to the field of nutrition and body composition (human somatotype), and more particularly, to systems, methods, and apparatuses for implementing a cloud-based health, nutritional, and body composition analysis platform, in which models are utilized to provide personalized data for health and nutritional counseling.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to embodiments of the claimed inventions.

Physical activity, body composition, and nutrition are key foundations of human health. These foundations are particularly important for patients with metabolic-related disorders such as obesity and diabetes, or even healthy individuals seeking to improve their cardiovascular fitness and body composition through the reduction of visceral fat.

Problematically, human health and nutritional assessment for risk and therapeutic purposes are highly dependent on the characteristics of the individual patient. Current weight loss methods focus on a weight-centered approach targeting changes in biometrics such as body mass index (BMI), waist circumference, and weight to screen and analyze metabolic-related disorders and associated risk factors. While these biometrics are useful for epidemiologic studies, when applied to assess the health status of patients they lack sensitivity and specificity. Furthermore, these commonly-used biometrics have limited utility in guiding health professionals in making clinical decisions. Most importantly, such a weight-centered approach cannot provide critically important information about the staging of the health risks of individual patients, hence they cannot be used to prevent and monitor the evolution of these risks in a timely fashion.

What is needed is a body-centered or anthropometric approach to health and nutrition analysis that simplifies the nutritional assessment of individuals while being uniquely suited for diffusion on a very large scale due to the recent rapid rise in telemedicine and online nutritional consulting caused by the Covid-19 pandemic.

The present state of the art may therefore benefit from the systems, methods, and apparatuses for implementing a cloud-based health, nutritional, and body composition analysis platform as set forth herein.

### BRIEF DESCRIPTION OF THE DRAWINGS

Embodiments are illustrated by way of example, and not by way of limitation, and can be more fully understood with reference to the following detailed description when considered in connection with the figures in which:

FIGS. 1A and 1B illustrate an exemplary user interface for health, nutritional, and body composition analysis, in accordance with described embodiments;

FIG. 2 illustrates exemplary body metric measurements for body circumference, in accordance with described embodiments;

FIG. 3 illustrates exemplary body morphs composition types, in accordance with described embodiments;

FIGS. 4A and 4B illustrate an exemplary smart analysis that may be used for online nutritional consultations, in accordance with described embodiments;

FIG. 5 illustrates an exemplary complete analysis that may be used for outpatient visits, in accordance with described embodiments;

FIG. 6 illustrates an exemplary table (Table 1) describing physical activity level score based on a number of daily steps taken, in accordance with described embodiments;

FIG. 7 illustrates an exemplary table (Table 2) describing height, neck, and size classification based on percentile, in accordance with described embodiments;

FIG. 8 illustrates an exemplary table (Table 3) describing classification of Body Mass Index (BMI), in accordance with described embodiments;

FIG. 9 illustrates an exemplary table (Table 4) describing categorization of percent of body fat between sexes, in accordance with described embodiments;

FIG. 10 illustrates an exemplary table (Table 5) categorizing body distribution based on waist-hip ratio for both sexes, in accordance with described embodiments;

FIG. 11 illustrates an exemplary table (Table 6) classifying excess abdominal volume, in accordance with described embodiments;

FIG. 12 illustrates an exemplary table (Table 7) determining metabolic syndrome risk score based on waist circumference for both sexes, in accordance with described embodiments;

FIG. 13 illustrates an exemplary table (Table 8) determining metabolic syndrome risk score based on waist/hip ratio for both sexes, in accordance with described embodiments;

FIG. 14 illustrates an exemplary table (Table 9) determining metabolic syndrome risk score based on waist-height circumference ratio for both sexes, in accordance with described embodiments;

FIG. 15 illustrates an exemplary table (Table 10) classifying individual metabolic syndrome risk based on a risk score, in accordance with described embodiments;

FIG. 16 illustrates an exemplary table (Table 11) classifying cardiovascular risk based on waist circumference for both sexes, in accordance with described embodiments;

FIG. 17 illustrates an exemplary table (Table 12) providing adiposity/muscularity risk classification based on age and sex, in accordance with described embodiments;

FIG. 18 illustrates an exemplary table (Table 13) describing physical activity level (PAL) based on physical activity level scores, in accordance with described embodiments;

FIG. 19A illustrates an exemplary table (Table 14A) describing normal body fat percentage by age group between both sexes, in accordance with described embodiments;

FIG. 19B illustrates an exemplary table (Table 14B) describing coefficients for personalized physiological weight based on lean mass estimate, in accordance with described embodiments;

FIG. 20 illustrates an exemplary table (Table 15) describing protein intake multiplication factors and contexts in which to apply them, in accordance with described embodiments;

FIGS. 21 and 22 depict flow diagrams illustrating methods for implementing a cloud-based health, nutritional, and body composition analysis platform, in which models are utilized to provide personalized data for health and nutritional counseling, in accordance with disclosed embodiments;

FIG. 23 shows a diagrammatic representation of a system within which embodiments may operate, be installed, integrated, or configured; and

FIG. 24 illustrates a diagrammatic representation of a machine in the exemplary form of a computer system, in accordance with one embodiment.

### DETAILED DESCRIPTION

Described herein are systems, methods, and apparatuses for implementing a cloud-based health, nutritional, and body composition analysis platform, in which models are utilized to provide personalized data for health and nutritional counseling. The system described herein, Morphogram, includes: a memory to store instructions; a processor to execute the instructions; in which the instructions are configured such that, when executed by the processor, the system carries out operations including: issuing a prompt from the system to transmit a GUI to a user device for display to a user, in which the GUI contains instructions to manually measure body metric measurements, receiving, at the system, user input transmitted to the system from the GUI at the user device providing one or more of (i) body metric measurements, (ii) medical, social, and dietary history, for a patient, and (iii) pedometer data.

Optionally, the Morphogram Platform may additionally integrate with data from wearable devices and biomarkers (e.g., such as for SM diagnosis). Additionally, the Morphogram Platform may be integrated with technologies that use the mobile camera for the detection of the body measures so as to capture a patient's body circumferences measurements.

As set forth herein, the Morphogram Platform additionally calculates anthropometric indicators of central body fat mass based on comparing the body metric measurements to age-group percentiles; determines a physical activity level; determines a body type (e.g., a constitutional biotype based on one or more of: (i) height. (ii) neck circumference, (iii) size, and (iv) waist-to-height ratio; calculating a physiological lean body mass and percentage of body fat; calculates a target physiological weight. Pursuant to the captured data, operations and calculations, the Morphogram Platform then outputs a GUI to a display of the user device to display a personalized risk monitoring profile for the patient based on the user inputs, determined factors, and calculated factors, including one or more of: (i) a complete nutritional status assessment, and (ii) a self-monitoring and maintenance assessment, in which the complete nutritional status assessment is based on a greater number of body metric measurements than the self-monitoring and maintenance, in which the personalized risk monitoring profile is utilized in achieving the target physiological weight.

The extraction of anthropometry-based biometrics is non-invasive, simple to execute, portable, low cost, and based on reference values obtained from peer-reviewed scientific literature. Therefore, this approach for evaluating the individual's nutritional status in terms of lifestyle, body composition and metabolic health risks evaluation is grounded on rigorous scientific data. Despite these advantages, anthropometry-based biometrics have been largely neglected in clinical practice in favor of a bodyweight-centered approach, or they have been used in very different ways. As a result, to date no unified system or process to interpret anthropometry-based biometrics to drive clinical decisions about nutritional and physical exercise prescriptions exists. These gaps led to a standard and scientific methodology to create an integrated and effective assessment of the nutritional status of clinical populations and healthy individuals. According to certain embodiments, the methods, systems, and apparatuses described herein are known as MORPHOGRAM or the MORPHOGRAM platform.

The methodologies and advancements embodied within the MORPHOGRAM platform are the result of clinical experience, with the aims of making nutritional status assessment simple and accessible to all and offering a practical, reliable method of obtaining and interpreting data leveraging criteria extracted from peer-reviewed scientific literature. According to certain embodiments, MORPHOGRAM may be offered as a subscription service for nutritional professionals to analyze and visit patients in person or remotely. According to other embodiments, MORPHOGRAM may be a mobile application for personal use. MORPHOGRAM is an easy-to-use tool for the prevention of weight-related diseases and allows remote monitoring of patients' health status by nutritional professionals. MORPHOGRAM takes into account various health-related and clinical factors and processes, such as: anamnesis (a patient's account of his or her medical history), lifestyle, clinical conditions, eating habits, nutritional status assessment, energy needs and expenditure, body composition, body type, health risk factors, nutritional personalization, customized nutritional plans, follow-up, and diet plans.

Clinical conditions may include food allergies (celiac disease, lactose, intolerance, nickel intolerance), metabolic conditions (diabetes, metabolic syndrome, development obesity, eating disorders, malnutrition, sarcopenia, osteoporosis, venolymphatic insufficiency), gastrointestinal disorders (gastroesophageal reflux, diarrhea, constipation, abdominal swelling or meteorism), sleeping disorders (insomnia, sleep apnea), female-related conditions (oligomenorrhea/amenorrhea, menometrorrhagia, infertility, pregnancy, feeding time, menopause), co-morbidities (allergies, autoimmune disorders, cardiovascular, dermatological, endocrine, gastrointestinal, neuropsychological, oncological, respiratory, urological), clinical interventions (accidents, surgical interventions, pharmacological therapies, asthenia or abnormal physical weakness or lack of energy), eating habits and schedule (including working times and shift work times, sport activities, alcohol use, tobacco use, and dietary restrictions such as vegetarian and vegan), nutritional status assessment, and anthropometric data and indicators.

The widespread diffusion of weight-loss programs and non-medical professionals such as personal trainers and nutrition coaches managing patients has pushed the medical assessment industry towards a greater focus on weight rather than an integrated overview of all clinically-relevant dimensions which is necessary for not only disease assessment but also disease prevention. Commonly used biomedical tools for body composition analysis (i.e., bioimpedance) have significantly reduced nutritional status assessment procedures to a simple body composition evaluation in terms of FFM, FM and Hydration. This superficial assessment is commonly based on an instrumental analysis, made with electrodes, rather than an integrated assessment that should address the analysis of body variation (i.e., body volumes, the ratio between height and specific body circumferences, fat distribution), through anthropometry, for the evaluation of health risks.

MORPHOGRAM analysis is entirely based on a body-centered approach, based on anthropometry, with a particular focus on central adiposity and provides an integrated analysis of cardiovascular risk, sleep apnea risk, metabolic syndrome risk, and body composition. Moreover, it introduces a new exclusive parameter called “Abdominal Volume Excess” which allows a more sensitive staging of central adiposity and provides a better understanding of patient status. This information can be used to lead nutritional and fitness professionals to precise, user-centric, and data-driven design of nutritional and physical exercise programs.

Thus, MORPHOGRAM is an analytical method designed as a digital tool for nutrition professionals and personal use. MORPHOGRAM promotes a body-centered approach, focused on prevention that allows remote management of patient health status to enable self-monitoring of weight-related diseases and analysis of body composition.

In the following description, numerous specific details are set forth such as examples of specific systems, languages, components, etc., to provide a thorough understanding of the various embodiments. It will be apparent, however, to one skilled in the art that these specific details need not be employed to practice the embodiments disclosed herein. In other instances, well-known materials or methods have not been described in detail to avoid unnecessarily obscuring the disclosed embodiments.

In addition to various hardware components depicted in the figures and described herein, embodiments further include various operations which are described below. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

The algorithms and displays presented herein are not inherently related to any particular computer or other apparatuses. Various customizable and special purpose systems may be used with programs in accordance with the teachings herein, or it may prove convenient to construct more specialized apparatus to perform the required method steps. The required structure for a variety of these systems will appear as set forth in the description below. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

In addition to various hardware components depicted in the figures and described herein, embodiments further include various operations which are described below. The operations described in accordance with such embodiments may be performed by hardware components or may be embodied in machine-executable instructions, which may be used to cause a special-purpose processor programmed with the instructions to perform the operations. Alternatively, the operations may be performed by a combination of hardware and software, including software instructions that perform the operations described herein via memory and one or more processors of a computing platform.

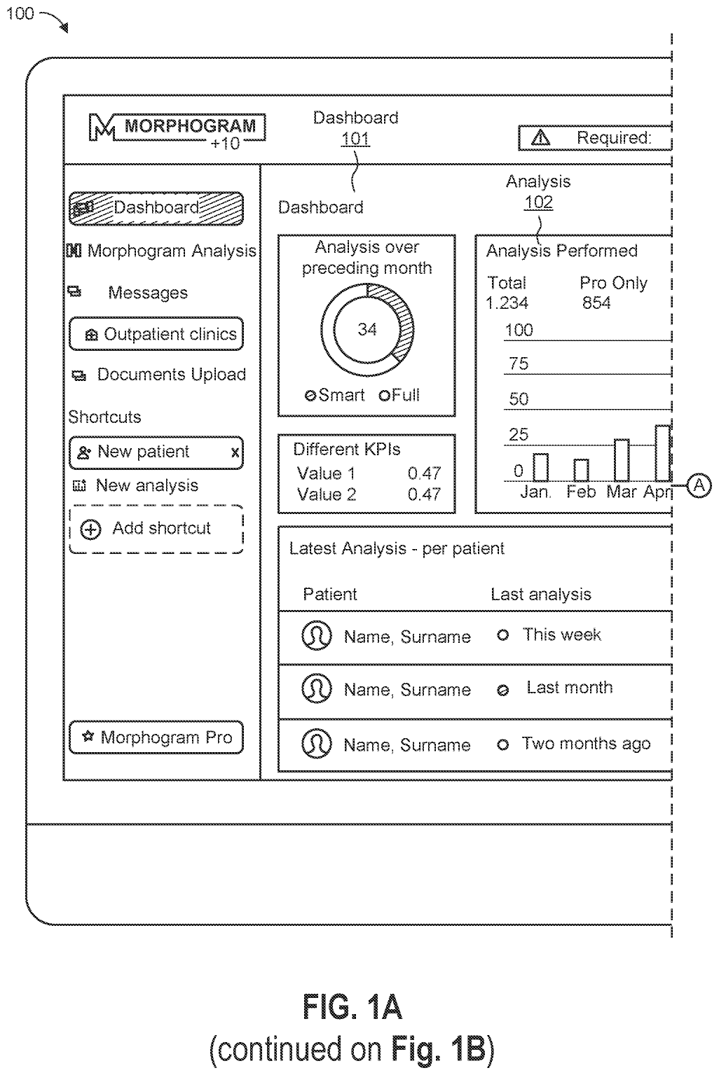

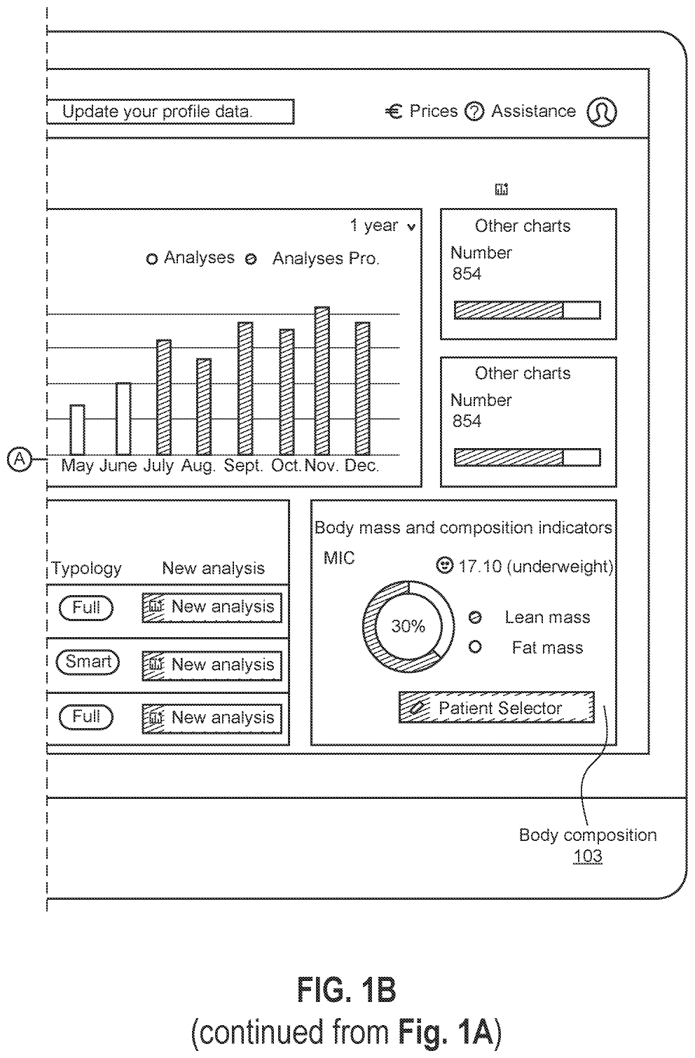

FIGS. 1A and 1B illustrate an exemplary user interface for health, nutritional, and body composition analysis, in accordance with described embodiments.

As shown here, MORPHOGRAM user interface 100 may include a dashboard 101 (FIG. 1A) highlighting various outputs based on user inputs such as body metric measurements, health history, and other parameters. Analysis section 102 (FIG. 1A) may provide information about the number and type of nutritional/health analysis performed using MORPHOGRAM. Body composition section 103 (FIG. 1B) may illustrate various body composition percentages including physiological lean mass, body fat mass, and body-mass index (BMI).

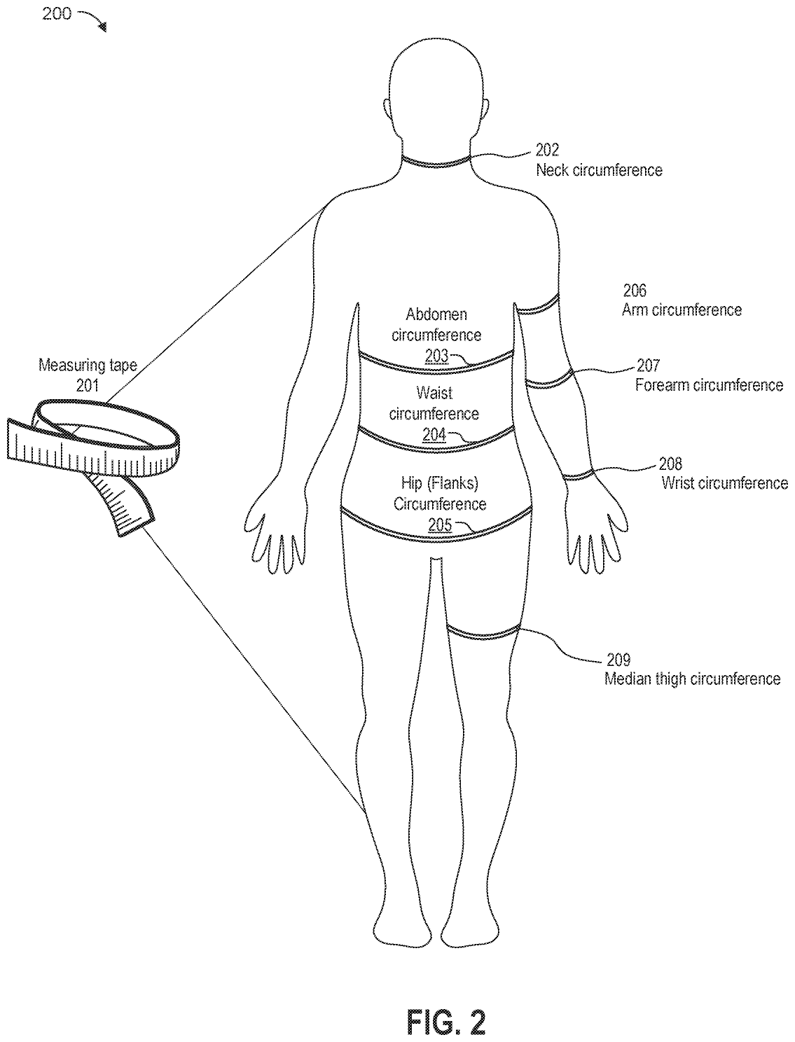

FIG. 2 illustrates exemplary body metric measurements for body circumference 200, in accordance with described embodiments.

A strategy focused on the pursuit of a patient's well-being should aim at reducing weight-related health risks (i.e., cardiovascular diseases, metabolic disease, cancers, sarcopenia, eating disorders). Therefore, nutrition professionals need to know about the staging of possible health risks to choose the right approach on a patient-specific basis.

This can be accomplished through a simple and reliable method, which consists of patient health history data and additionally measuring the circumference at specific points on the body and analyze their relations with height, body composition, and body type.

Body metric measurements may be taken for a patient in a clinical setting or self-performed by the patient or an assistant remotely and entered as user input into the MORPHOGRAM platform. Measuring tape 201 is a simple and effective way to take such measurements at various circumferences on the patient's body. In addition to height in centimeters and weight in kilograms, MORPHOGRAM requires measurements acquired with easy and appropriate standardization criteria in both males and females via measuring tape 201. These measurements include common body circumferences such as neck circumference 202, waist or abdominal circumference 204, hip or flank circumference 205, arm or mid-arm circumference 206, forearm circumference 207, wrist circumference 208, and median thigh circumference or median thigh circumference 209. These body circumference measurements may provide an estimate of body fat, including visceral fat and subcutaneous fat, when combined with height and weight. Using these body measurements, body analysis is made including constitutional indicators (body type, size, fat distribution), body mass and body composition indexes (body mass index (BMI), fat free mass index (FFMI), fat mass index (FMI), fat free mass and fat mass metrics (fat free mass in kilograms, fat mass percent), risk factor metrics (waist circumference in centimeters, waist-to-height ratio (WHtR), waist-hip ratio (WHR), neck circumference in centimeters, neck-height ratio (NHR), waist-thigh ratio (WTR), and lean mass functionality indicators such as handgrip force.

These data points are collected through a simple, quick, low-cost, and standardized procedure and provide scientifically validated information. Data measurements are integrated with medical history information including clinical assessment, comorbidities, food allergies, eating habits (including vegetarian and vegan diets), and lifestyle as measured by physical activity level (PAL).

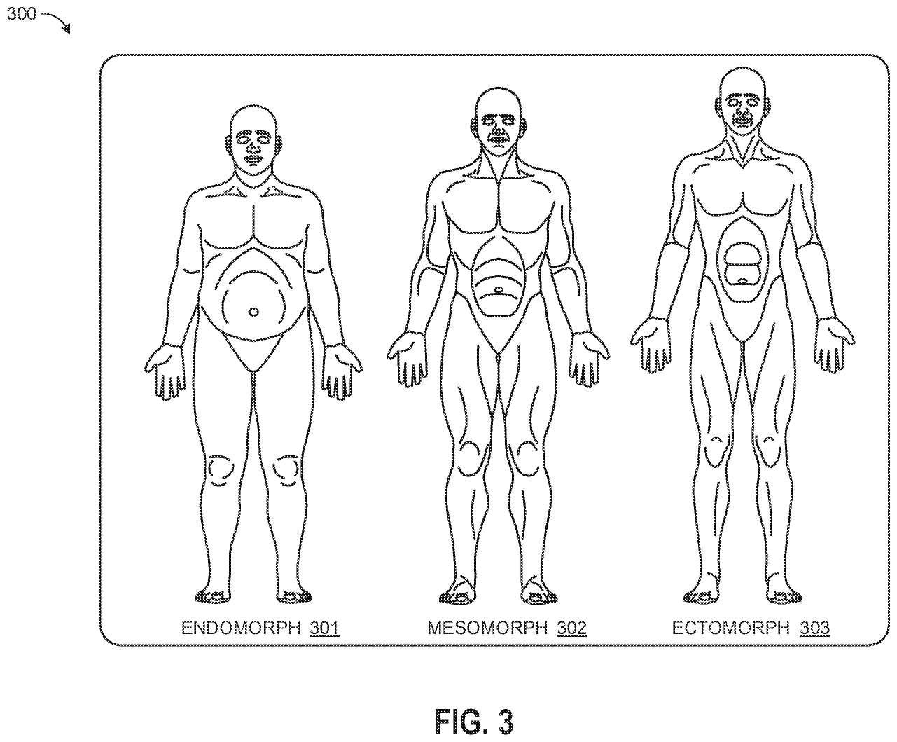

FIG. 3 illustrates exemplary body morphs composition types, in accordance with described embodiments.

The organization of anthropometric measurements used, according to the percentiles of the respective age groups (from 5th to 95th percentiles), gives the possibility to evaluate the extent to which the body is in a balanced or unbalanced state. The processing of these measurements outlines, for each subject examined, a direct and indirect reading of the morpho-functional characteristics, through the following indicators: constitutional indicators, body mass, and body composition indicators, fat free mass indicators, fat mass indicators, risk factors indicators, and lean mass functionality indicators (handgrip).

### Fat Free Mass (FFM) and Fat Mass (FM) Assessment.

The estimation obtained using anthropometric methods is fundamental in the epidemiological and clinical fields, bearing in mind that the equations that include the combination of waist circumference and waist circumference relative to the hips circumference explain 91% of the variation in the total volume of adipose tissue.

Analysis of clinical samples and constant verification with other methods was essential to select the most appropriate equations. The present methodology focused on the equations that most sensitively detect changes in abdominal-visceral fat and gluteal-femoral/subcutaneous fat, taking into account the variables related to age and constitutional factors.

Equations were chosen that are most sensitive to changes in the circumferences of the waist, abdomen, and hips, to support the goal of preventing, monitoring, and reducing the risk or consequences of obesity and metabolic syndrome.

This allows for a prediction consistent with the anthropometric and clinical survey, although with the awareness of the fact that these predictive equations are less reliable in some types of subjects. Specifically, an underestimation of lean mass and overestimation of body fat may occur in older age groups and with body mass indices below 18.5, or in the long-limbed, while in obese subjects with BMI over 40, an underestimation of body fat, in the latter case, also due to the evident difficulty in measuring the increase in fat from the hanging abdomen.

A final critical issue is given by subjects in which the only measurement of circumferences may not adequately estimate the subcutaneous/gynoid fat. In these cases, it is also useful to perform the skin test, to avoid an overestimation of the lean mass.

### Equations

The equations used by MORPHOGRAM to estimate lean mass and the percentage (%) of body fat are the following:

### Lean Equation.

This equation takes into account age and waist circumference to estimate the percentage of body fat in a various range of ages:

$$Male : 0.567 \times waist + ( 0.1 \; 01 \times age ) - 31.8 \\ Female : 0.439 \times waist + ( 0.221 \times age ) - 9.4$$

The equation is not used in underweight and normal weight subjects between 18 and 25 years old and in athletes.

### Relative Fat Mass (RFM) Equation.

This equation is based on the height-to-abdomen ratio:

$$Male : 64 - ( 20 \times height / abdomen ) \\ Female : 76 - ( 20 \times height / abdomen )$$

The equation is not used in underweight and normal weight subjects between 18 and 25 years old and in athletes.

### Body Adiposity Index (BAI).

BAI based on the hips-to-height ratio:

$$BAI : Hips / ( Height \times √ ( Height ) ) - 18$$

The equation is not used in underweight and normal weight subjects between 18 and 25 years old and in athletes.

BAI estimates the percentage of body fat starting from the principle that this correlates positively with hips circumference, while it is negatively correlated with height.

### Hodgdon and Beckett Equation.

This equation is used for the measurement of fat mass (FM) % in men and includes height, neck and abdomen and takes in count those measures which are related to lean mass (height and neck), with abdominal circumference which is connected with fat mass:

$$Density = - 0.191 \times Log 10 ( abdomen - neck ) + 0.155 \times Log 10 ( height ) + 1.032 \\ \% FM = 100 \times [ ( 4.95 / density ) - 4.5 ]$$

The equation is used in underweight and normal weight subjects between 18 and 25 years old and in athletes.

### Vogel Equation.

This equation is used for the measurement of fat mass % in women and includes height, weight, neck, forearm, wrist, and hip and takes into account height, neck, wrist, and forearm as lean mass indicators while considering hips and weight as fat mass indicators:

$$\% FM = 105.3 \times Log 10 \times weight - 0.2 \times wrist -$$

$$0.533 \times neck - 1.574 \times forearm + 0.173 \times hips - 0.515 \times height - 35.6$$

The equation is used in underweight and normal weight subjects between 18 and 25 years old and in athletes.

### MORPHOGRAM Algorithm.

The MORPHOGRAM formula uses for the first time the weighted average of two equations (BAI and RFM) which both estimate adiposity, but while the first calculates it from the value of hip circumference in relation to height, the second calculates it from the value of height in relation to the abdominal circumference. The Lean equation has also been added to MORPHOGRAM's algorithm, which also integrates the waist circumference and age.

Through numerous clinical, anthropometric, and impedance tests we have created the following MORPHOGRAM algorithm:

$$\% FM : ( [ ( RFM + BAI ) ] + Lean ) / 2$$

### Constitutional Indicators.

The constitutional characteristics (e.g., body type indicators) are particularly important because they influence not only the body image, but also the consistency of the skeletal framework and lean mass, and attention has been paid to the fact that the constitution and physiological activity are closely correlated.

Body type definitions allow for a deeper knowledge of the body and of the endocrine, neuro-vegetative, and metabolic peculiarities belonging to each morphotype.

Regarding the definition of the main body types, MORPHOGRAM uses an algorithm that integrates the following parameters: height, neck circumference, size (height-to-wrist ratio), and waist-to-height ratio (WHtR).

The morpho-functional classification adopted also makes it easier to understand how there is an enormous structural variability within the weight normality that we usually consider in the BMI range of 18.5 to 24.9.

MORPHOGRAM's algorithm for the definition of the constitutional biotypes is based on a morpho-functional classification and defines four fundamental clusters of biotypes: Ectomorph 303, Leptosomic, Mesomorph 302, and Endomorph 301.

Distinctive characteristics have been indicated for intermediate biotypes, attributing the general characteristics to the most representative biotype.

In addition, the algorithm also provides for the identification of endomorphism acquired in all constitutional biotypes that can meet, through an increase in central adiposity, an increase in the waist-to-height ratio (WHtR)>0.5. The above-mentioned data is important because it provides an early warning to possible cardio-metabolic risk to which the subject is exposed.

The endomorph biotype 301 is an exception, in which endomorphism is constitutional, but the reference of the cut-off of the waist-to-height ratio (WTR)>0.5 remains useful both for the identification of the biotype and for the prevention of the further increase of the risk that becomes significant for values above 0.6.

In practice, subjects with a BMI between 18.5 and 24.9 are classified as having normal weight. However, thanks to the identification of constitutional biotypes and the algorithm used by MORPHOGRAM, we find that the long-limbed-ectomorphs 303, which from a morpho-functional point of view we can indicate as “thin”, have their normality in a BMI range between 18.5 and 20.5. Leptosomic and mesomorphs 302, which we can indicate as “balanced”, have their normality in a BMI range between 20.6 and 23.5.

Finally, we will find that the short limbed-endomorphs 301, which we can indicate as “abdominal.” due to the prevalence of the abdomen in comparison to the chest in these individuals, have their normality in a BMI range between 23.6 and 24.9, but are distinguished by a waist-to-height ratio (WHtR)>0.5. Those that we define as “muscular.” due to the prevalence of lean mass, include different mesomorphic phenotypes 302 which, although falling within the same range of BMI, have a waist-to-height ratio <0.5. However, there are also mesomorphic phenotypes 302 which we define as “subcutaneous” because they can present a reduced lean mass and an excess of subcutaneous fat and furthermore, especially in women, an excess of gluteus-femoral fat.

These subjects can derive useful information on body composition from plicometry (the estimation of body mass by measurements of subcutaneous adipose tissue), to avoid drawing erroneous conclusions about body composition based only on circumferences.

These references will be useful, as we shall see, for the identification of physiological lean mass in underweight, normal weight, and overweight subjects, while avoiding attributing underestimated or overestimated physiological weight targets with respect to the constitutional biotype.

Finally, once the constitutional biotype has been identified, the cut-off of the waist-to-hip ratio (WHR) differentiates the distribution of fat in the android, gynoid, and intermediate morphotypes, further characterizing the constitutional biotype.

The android morphotype has fat distribution in the nape, neck, shoulders, chest, upper abdomen and has greater metabolic and vascular risks.

The gynoid morphotype, more typical of women, has a greater tendency to subcutaneous fat, with a predominant distribution on the hips, buttocks, thighs, and lower abdomen. It has a reduced metabolic and vascular risk, but weight overload and regional increase in fat can lead to osteoarticular and venolymphatic complications.

The intermediate or balanced morphotype has a mixed type of fat distribution.

The android, gynoid, or intermediate characterization concerns exclusively the distributive typology of fat and coexists with the constitutional biotypes described.

Below are the characterizing aspects of the main constitutional biotypes:

Long-limbed ectomorph 303 describes a basically frail subject identified very well with four selected distinctive characteristics: height >90th percentile, thin neck, slim size, and WHtR<0.5.

The long-limbed ectomorphs 303 have a prevalence of the chest over the abdomen and have long and thin limbs with little muscle. They tend to be hyper-sympathetic, catabolic, and easily oxidize fats. They have a predominance of the central and peripheral nervous systems that are derived from the ectodermal sheet. They have strong cerebral and creative activity but tend to overload the nervous system excessively and incur psycho-neuro-endocrine imbalances. They have a certain digestive and intestinal fragility that can produce a lack of appetite, dysbiosis, and malabsorption associated with subclinical inflammatory states. The resulting loss of lean mass and mineral density is the cause of sarcopenia, osteoporosis, and osteoarticular problems. To prevent loss of lean mass and osteoporosis, they must select appropriate nutritional methods and pursue physical activity that does not expose them to contact injuries. Their natural propensity is above all for those activities that require agility.

Ecto-mesomorph is the biotype that associates high stature with an adequate musculoskeletal structure that makes it metabolically belong to the normo-limbed-mesomorph.

Short-limbed leptosomic is the biotype that is characterized by short stature (height<10th centile) and slender body structure (small neck, slim size, short and thin arms). It is more harmonious than the long-limbed but finds it difficult to manage its metabolic balance and maintain body harmony over time. It is not considered as a constitutional biotype in its own right, but it is being taken into consideration here for morpho-functional reasons, as it has a reduced energy-nutritional requirement, compared to the average of the population, and is particularly exposed due to its body size. This exposure is due not only to metabolic overload, but also to nutritional imbalances. For this reason, the short-limbed-leptosomic should be referred to in the search for proper nutrition and the right balance between food and body type to promote a healthy, realistic, and stable body identity.

Short-limbed mesomorph is the biotype that is characterized by short stature and the predominance of the musculoskeletal system which makes it metabolically similar to the normo-limbed mesomorph.

Nutritional monitoring is particularly useful, both to prevent weight changes, and to avoid the easy increase of abdominal-visceral fat with the related cardio-metabolic-inflammatory risks.

Normo-limbed mesomorph has medium height (falling between the 25th and 75th percentiles), medium neck, and harmonious skeletal, connective tissue, and muscle development, with homogeneous percentiles of lean mass indicators and with abdomen not prevalent compared to the chest. Some mesomorphs 302, due to adaptive phenomena to work or sporting activities, may present muscle prevalence of the upper or lower limbs. Metabolic and cardiovascular pathologies appear later than in the normo-limbed mesomorph, often associated with significant obesity, joint, and vascular overload pathologies.

Mesomorph 302 appears, to all intents and purposes, as a point of balance between the long-limbed and endomorph biotypes. The mesomorph has good vitality and a remarkable ability to adapt to stress which improves its resistance, making it a good worker. Physicality and the ability to fully express it are the foundation of the health of the mesomorph which, in the absence of regular physical activity tends to accumulate abdominal fat.

The mesomorph is suited for all activities that require strength, agility, and speed. The increase in abdominal-visceral fat, however, activates the systemic inflammatory stimulus early through hypercortisolemia and hyperinsulinemia and rapidly leads to metabolic syndrome and related vascular complications.

Meso-ectomorph is the biotype of medium height and slender body structure that behaves metabolically like a long-limbed, from which it stands out for a more defined and centered physicality.

Short-limbed endomorph 301 is a short subject with a prominent abdomen identified as having: height<10th percentile, a short thick neck, a robust wrist in relation to height, and WHtR>0.5. The relative deficiency of the respiratory system contributes to the reduced oxidative capacity of fats in these individuals. The thickness of the subcutaneous fat reduces heat dispersion and facilitates hypometabolism which, associated with hypervagotonia, the prevalence of visceral organs, and hyperinsulinism, contributes to the anabolic and fattening tendency.

However, the natural tendency to gain weight is often underestimated because they are subjects who associate emotional stability and self-perception of a widespread state of well-being, also linked to good digestive efficiency, to good health. Thus, they may be emotional eaters.

The cut-off of WHtR>0.5 differentiates the endomorph 301 from the mesomorph 302 and allows to preventively counteract the tendency to gain weight with appropriate nutrition, regular physical activity, and periodic monitoring. In this biotype, the WHtR risk becomes significant for values>0.6. Endomorph 301 has a greater predisposition for activity in water, as well as activity involving strength and flexibility.

### Acquired Endomorphism.

Constitutional biotypes can modify the respective general metabolic-endocrine characteristics, due to the effect of acquired abdominal fat. When this involves an increase in the WHtR>0.5, “acquired endomorphism” results. This definition denotes the possible reversibility of the biotype, with a return to the constitutional biotype.

MORPHOGRAM identifies the following possibilities with acquired endomorphism:

Ecto-endomorph: We define “ecto-endomorph” as the long-limbed ectomorph which is fattened up to have a prominent and significant abdomen compared to height.

Nutritional monitoring is very useful for preventing or reducing metabolic and cardiovascular risk, which may be clinically more important in a biotype that is not constitutionally predisposed to increase central adiposity.

Lepto-endomorph: The lepto-endomorphic biotype is the short-limbed leptosomic which gains weight until it has a prominent and significant abdomen with respect to height and which, for this reason, acquires an increased risk of metabolic syndrome, also due to low lean mass levels.

Ecto-meso-endomorph: We define “ecto-meso-endomorph” as the ecto-mesomorphic biotype, characterized by high stature and robust musculoskeletal structure (metabolically mesomorphic), to which a prominent abdomen is added, increasing central adiposity. In sumo wrestling, this constitutional typology is fundamental, precisely because in this sport the abdomen acts as a center of gravity and represents strength. Nutritional monitoring is important to avoid both underestimation and overestimation of energy intake and to reduce or avoid the increase in abdominal adiposity.

Meso-endomorph: The “meso-endomorphic” biotype is the normo-limbed mesomorph which has gained weight, to the point of having a prominent and significant abdomen compared to height and which, for this reason, acquires an increased risk of metabolic syndrome.

Meco-ecto-endomorph: This is the biotype of medium height and slender body structure, metabolically long-limbed and with a prominent and significant abdomen compared to height, which causes it to acquire an increased risk of metabolic syndrome.

WHtR<0.5 discriminates between non-endomorphic constitutional biotypes (in which waist circumference does not represent a risk factor, in relation to height) and the short-limbed-endomorph, in which the WHtR is constitutionally >0.5.

According to certain embodiments, information provided by MORPHOGRAM is displayed as an easy-to-read health report for use by both medical and non-medical professionals as well as patients.

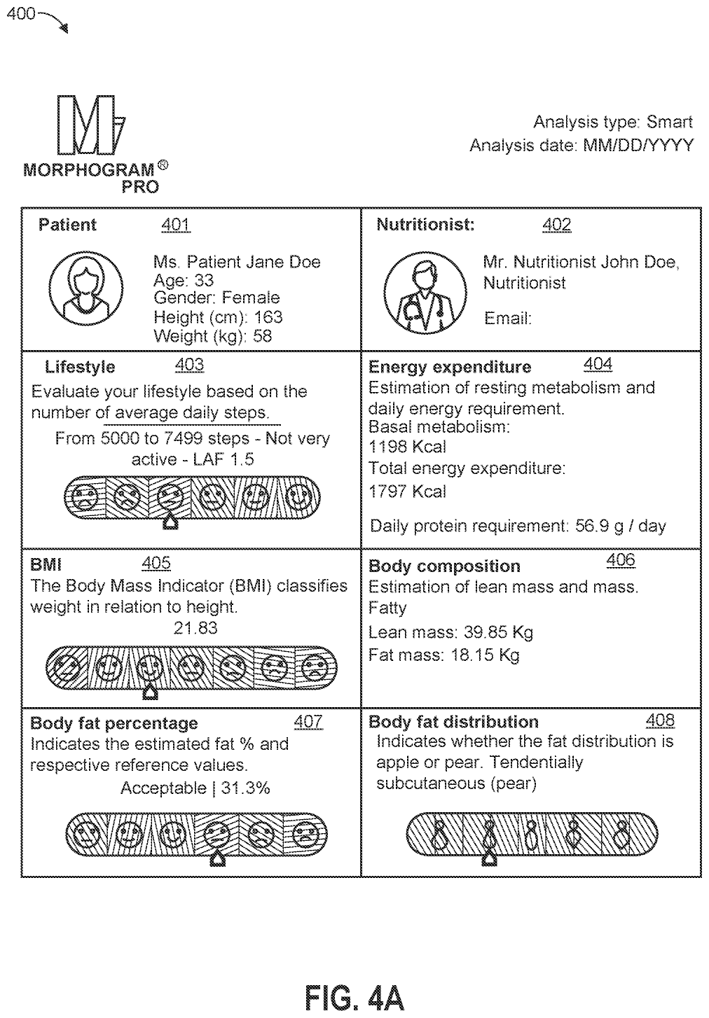

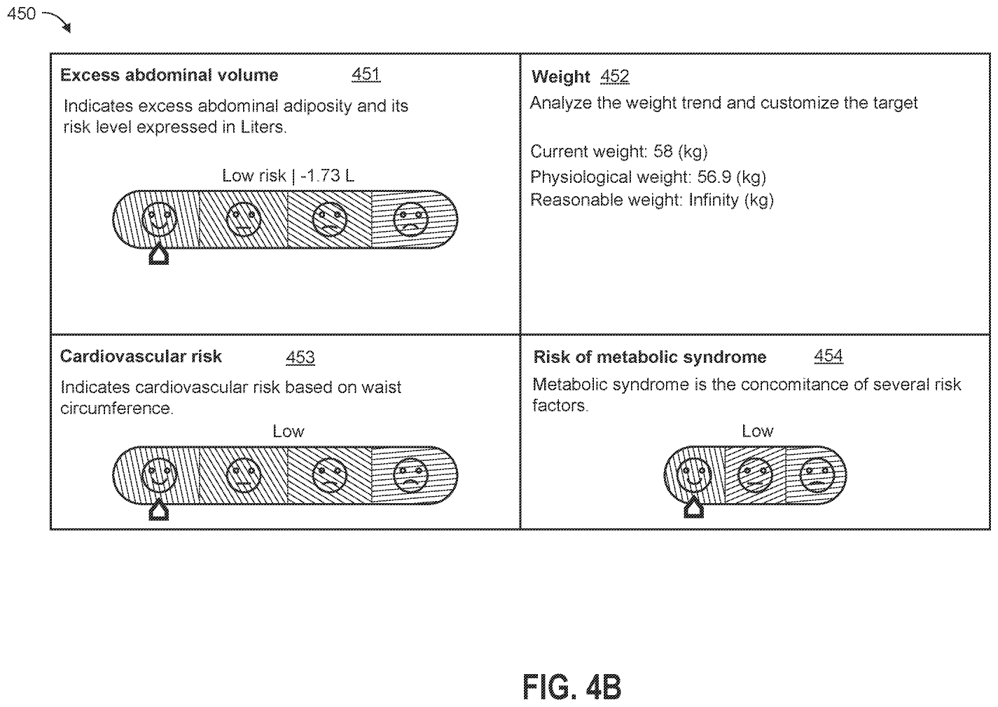

FIGS. 4A and 4B illustrate an exemplary smart analysis 400-450 that may be used for online nutritional consultations, in accordance with described embodiments.

As shown in FIGS. 4A and 4B, smart analysis 400-450 is a briefer report of nutrition and body evaluation, which may be used as a self-monitoring and maintenance assessment by a patient or clinician during follow-up visits or online consultation. According to certain embodiments, smart analysis 400-450 is based on five measurements. Smart analysis 400-450 begins with demographic and basic health analysis 400 at FIG. 4A which may include patient information 401, nutritionist information 403, physical activity level 403 (for example, as mentioned by the number of daily steps a patient takes), energy expenditure 404 including basal metabolic rate, daily energy, and protein requirements. Demographic and basic health analysis 400 further includes Basal Metabolic Rate (BMI) 405, body composition 406 including lean mass and fat mass, body fat mass 407, and body fat distribution 408.

FIG. 4B continues smart analysis 400-450 with excess abdominal volume 451, for example signifying a risk level expressed in liters. Weight 452 may provide various measures of weight such as actual weight, physiological weight, and reasonable weight. Cardiovascular risk 453 may indicate cardiovascular risk based on waist circumference. Finally, metabolic syndrome risk 454 indicates a patient's risk for metabolic syndrome, which is a constellation of risk or co-morbidities associated with obesity.

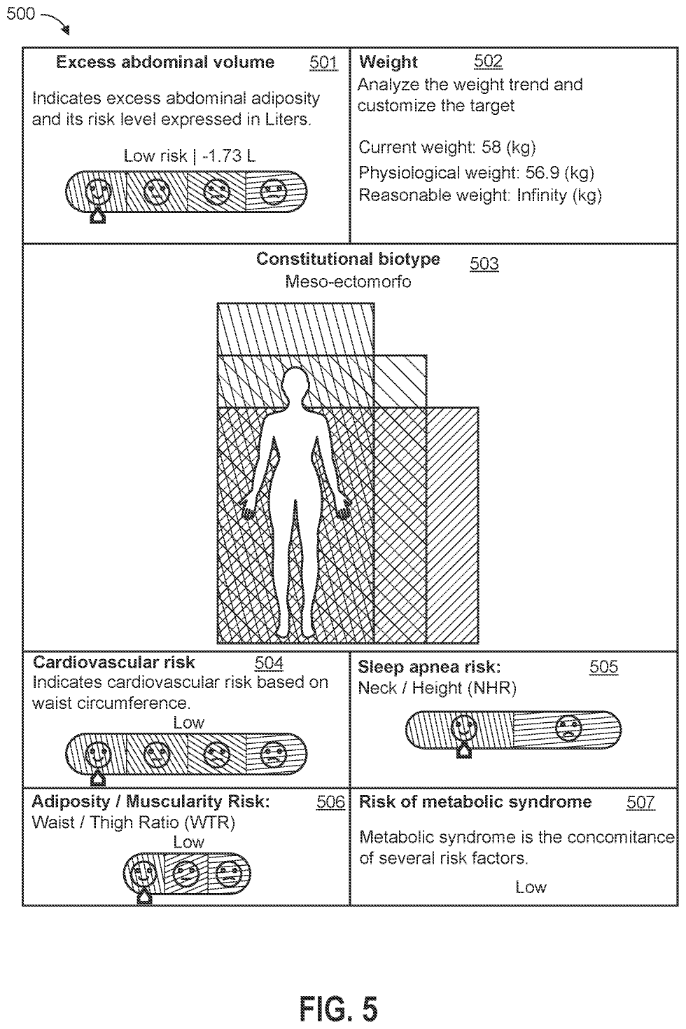

FIG. 5 illustrates an exemplary complete analysis 500 that is a full report used for a complete nutritional status assessment, for example during an initial visit, and outpatient visits, in accordance with described embodiments. According to certain embodiments, complete analysis 500 is based on ten measurements. Complete analysis 500 may including measurements and evaluations similar to smart analysis 400-450 but may additionally include a determination of a constitutional body type 503 for a patient, such as the body types described in FIG. 3. Complete analysis 500 may further include night apnea risk 50, which may be based on a neck-to-height ratio, as well as adiposity-muscularity risk 506, which may be measured based on the relationship between waist and thigh circumference.

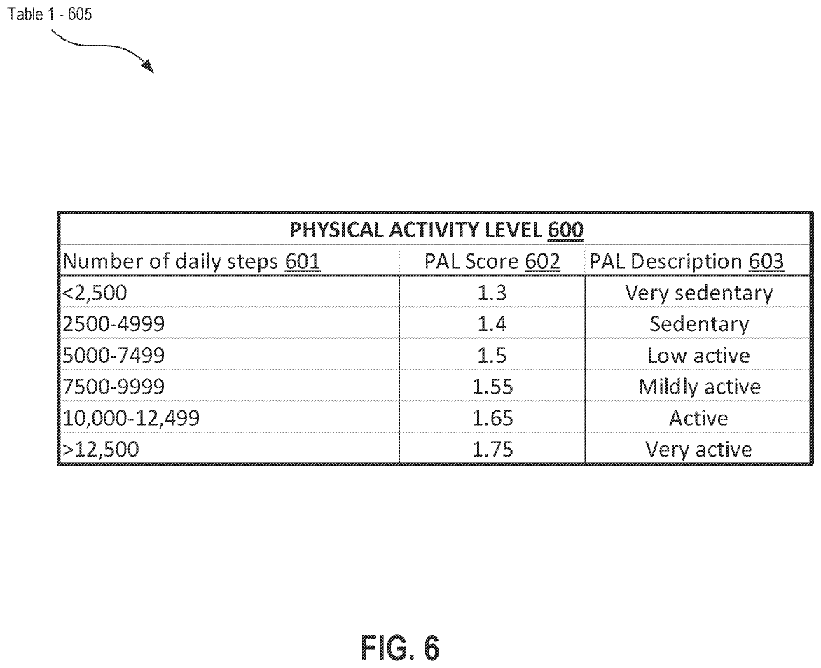

FIG. 6 illustrates an exemplary table (Table 1) describing physical activity level (PAL) score based on the number of daily steps taken, in accordance with described embodiments. As shown here, physical activity level (PAL) 600 is based on the number of daily steps 601 taken by an individual, with ranges of steps assigned a PAL score 602 that corresponds to a PAL description 603, which ranges from very sedentary (less than 2,500 steps taken per day) to very active (more than 12,500 steps taken per day).

Estimating PAL 600 is based on the number of steps 601 taken as measured by wearable devices such as a pedometer, smartphone, or smartwatch and is a validated methodology for evaluating a patient's lifestyle.

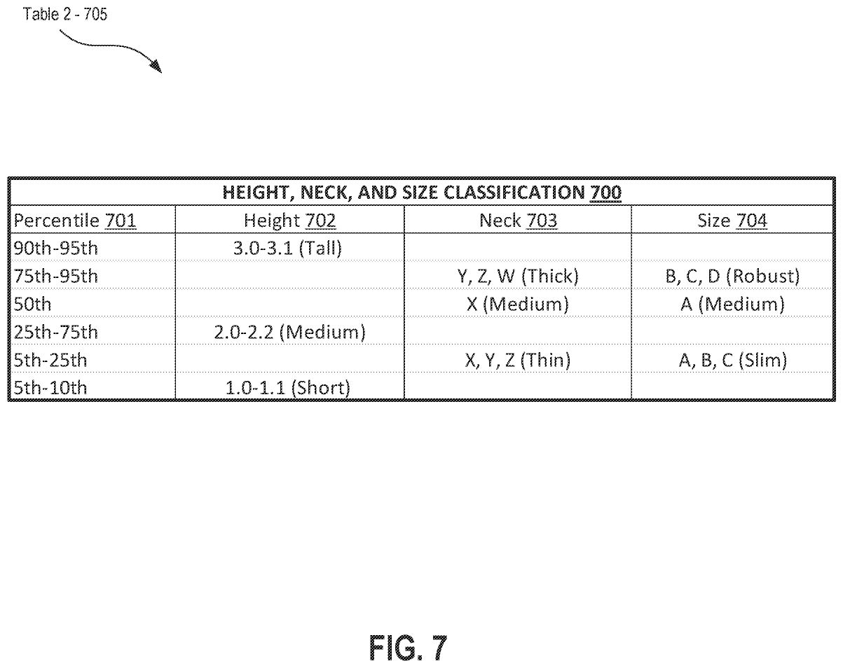

FIG. 7 illustrates an exemplary table (Table 2) describing height, neck, and size classification based on percentile, in accordance with described embodiments.

Height 702 ranges from short at the lowest measures of percentile 701 (such as the 5^{th}-10^{th} percentiles), to tall at the 90^{th}-90^{th} percentile, with medium height corresponding to the 25th-75th percentiles. Neck circumference 703 ranges from thin at the lower percentiles to thick at the higher percentiles. Body size 704 ranged from slim at the lowest percentiles to robust at the highest percentiles.

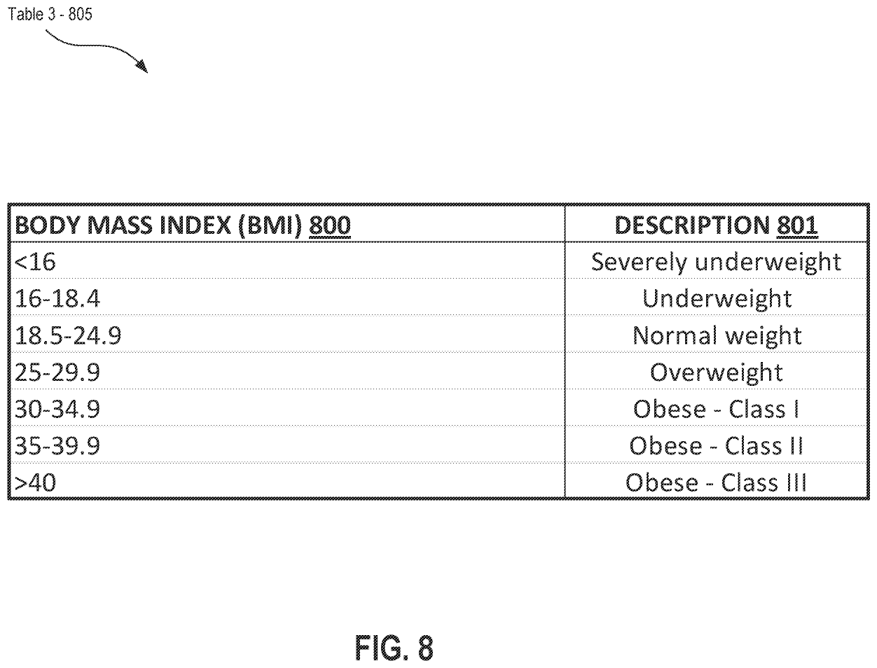

FIG. 8 illustrates an exemplary table (Table 3) describing classification of Body Mass Index (BMI) 800, in accordance with described embodiments. BMI 800 is a common measurement of body fat, calculated by a person's weight in kilograms divided by the square of their height in meters. BMI ranges from <16 which is described 801 as severely underweight to >40 which is categorized as Class III obese, with a normal weight considered to be a BMI between 18.5-24.9. BMI is used to separate the individual's body weight from the individual's height.

BMI, although widely used in clinical practice, does not provide information relating to body composition, as the same level of BMI can be found respectively in men and women who physiologically have different percentages of body fat and moreover, when considering the area of overweight and obesity, even subjects with a high degree of muscularity can be classified under the same level of BMI.

In addition, 11% of males and 6% of women are classified as “normal weight obesity”, which suggests that even with normal weight, there are also subjects with loss of lean mass and increases of fat. In this situation, it is of fundamental importance to evaluate the distribution of fat.

However, if BMI is completed with the assessment of body composition and central adiposity measurements, it becomes a very accurate risk predictor. We have already described the criterion used by MORPHOGRAM to estimate body composition, which starts from the principle of emphasizing changes in central adiposity.

Therefore, once the value in Kg of the lean mass (FFM) has been obtained, it is possible to calculate the Fat Free Mass Index (FFMI).

Likewise, from the value in Kg of the fat mass (FM), the Fat Mass Index (FMI) may also be calculated.

These indices are useful because they express the proportions of fat free mass and fat mass, within the body mass index which is the sum of the above (BMI=FFMI+FMI).

Previous studies reported percentiles of the FFMI and IMF values relating to the Italian population aged from 20 to 80 and, thanks to these percentiles, it is possible to evaluate the balance of lean and fat and all the conditions that may derive from the prevalence of one or the other.

For practical purposes, these indices give the possibility of recognizing among subjects classified respectively as normal weight, overweight or obese, those with balanced lean mass and fat mass (having FFMI and FMI in the same percentile), and those with a prevalence of lean mass (having FMMI a percentile higher than FMI), which we define as “stenics.”

It is necessary to distinguish the subjects with subcutaneous/gluteofemoral distribution in which, if the analysis is based on the circumferences alone, there is a risk of overestimating the lean mass, so in these cases, after having directly ascertained the consistency of the subcutaneous fat, it is recommended to integrate plicometric examination to improve the reliability of body composition.

Finally, those with lean mass reduction (who have FFMI one or more centiles lower than FMI) are defined as “sarcopenics”.

This distinction has its importance because the reduction of lean mass and the increase of fat mass are independent predictors of all causes of mortality and can also be predictors of metabolic syndrome.

Since these indices are derived from the estimation of lean mass, the absolute value is not so important as the trend highlighted, which is helpful in preventing and monitoring conditions of sarcopenia or sarcopenic obesity.

Finally, in underweight subjects and in subjects with BMI>40, MORPHOGRAM does not estimate FFMI and FMI.

In normal weight and underweight subjects with FFMI<FMI, the lean mass deficit can be assessed, as we shall see, by calculating the physiological lean mass.

### Fat Free Mass Indicators.

Selected indicators of lean mass include percentiles for arm circumference, median thigh circumference, and body weight by sex and by age group.

In contrast, lean mass percentiles were obtained from the respective percentiles of body weight less the percentage of body fat considered physiological by sex and by the respective age groups.

If the lean mass is on the same percentile as the weight, it means that the percentage of fat is adequate and that the lean mass is in proportion to the weight (justified weight). In this case, however, it will be appropriate to evaluate the harmonic distribution of the fat.

If, on the other hand, the lean mass is a percentile (or more) lower than the weight, in most cases it is an excess of fat mass, sometimes with a relative reduction in lean mass (sarcopenic obesity), but a selective reduction in lean mass (as in obese normal weight).

Finally, if the lean mass is about one centile higher than the weight, it means that the lean mass prevails over the fat.

The comparison between the percentiles of the arm circumference and median thigh circumference can highlight conditions of proportionality or conditions of prevalence of one over the other and vice versa.

The latter can have various interpretations and must be analyzed in a personalized way, also evaluating the patient's lifestyle and the prevalent physical activity that could favor, depending on the case, the upper or lower limbs.

The comparison between the percentiles of the arm circumference and/or the median thigh circumference with the percentiles of the lean mass and weight is useful for evaluating the proportion of the limbs with respect to the latter.

If all four parameters are consistent, we are dealing with a normo-limbed-mesomorphic biotype.

If the percentile of the median thigh circumference is consistent with the percentile of the lean mass but lower than the percentile of the weight, it indicates that the thigh is not proportionate in relation to the weight to be supported. In this condition, the muscles of the lower limb may be metabolically inefficient.

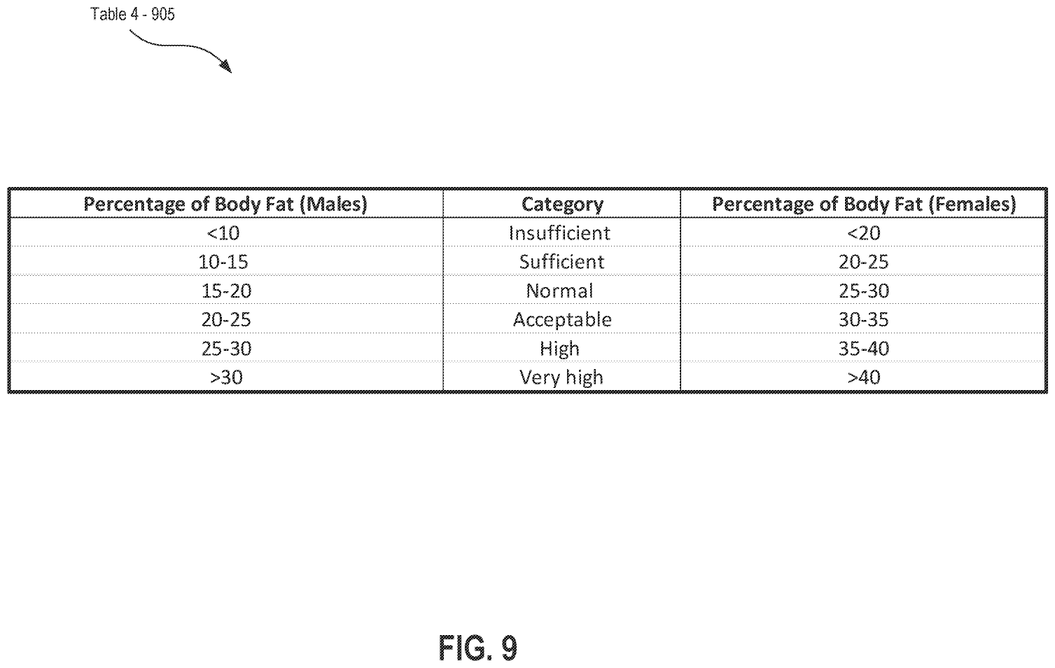

FIG. 9 illustrates an exemplary table (Table 4) describing categorization of percent of body fat between sexes, in accordance with described embodiments. Percentage of body fat is classified similarly between males and females, with less than 10% body fat considered as insufficient, 15-20% body fat considered normal, and more than 30% of body fat considered as being very high. Among the indicators of adiposity, the reference levels of the estimated percentage of fat, as a generic indicator of risk, are considered.

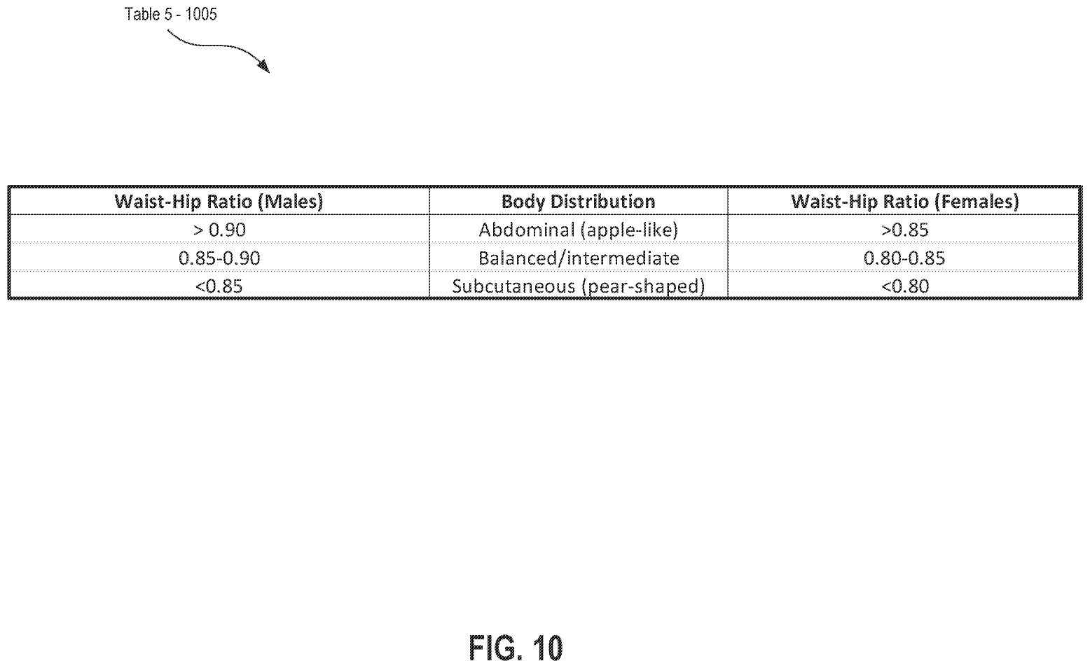

FIG. 10 illustrates an exemplary table (Table 5) categorizing body distribution based on waist-hip ratio for both sexes, in accordance with described embodiments. As shown here, waist-hip ratios for males and females are associated with a body distribution type. Women with a waist-hip ratio greater than 0.85 are considered to have an abdominal or apple-like body distribution, while men with a waist-hip ratio greater than 0.90 are considered to have this distribution. Thus, women have lower waist-hip ratio ranges for the same body type as men do. A balanced or intermediate body distribution for males is considered to be 0.85-0.90 while women must have a waist-hip ratio of 0.80 to 0.85 to be classified within a balanced or intermediate body distribution. A low waist to hip ratio (less than 0.85 for men and less than 0.80 for women) implies wider hips and a smaller waist implies a subcutaneous or pair-shaped body distribution.

### Excess Abdominal Volume (AV+).

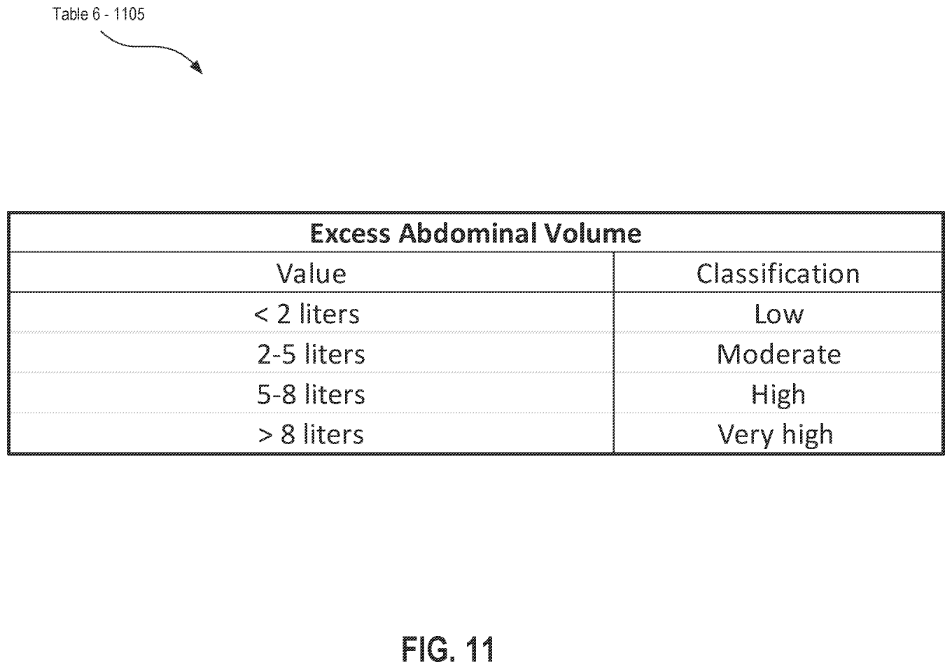

FIG. 11 illustrates an exemplary table (Table 6) classifying excess abdominal volume, in accordance with described embodiments. Excess abdominal volume measures the excess amount of volume that the abdominal area takes up and may be classified as low if less than two liters and very high if more than 8 liters, with 2-5 liters considered moderate excess abdominal volume. Excess abdominal volume allows for a more sensitive staging of central adiposity and provides for a better understating of patient health status. This information can be used by nutritional and fitness professionals to design precise, user-centric, and data-driven nutritional and physical exercise programs.

We will focus on the excess abdominal volume (AV+) which offers a further contribution for monitoring central adiposity and for quantifying individual risk.

The Excess Abdominal Volume (AV+) is a new and practical indicator that is calculated, in the subject under examination, when his waist circumference is greater than half of his height. Therefore AV+is not calculated in subjects with low-risk waist circumference.

This index, expressed in liters, gives a clear volumetric reference useful for grading and sizing excess abdominal adiposity and its level of risk.

To understand the meaning of the Excess Abdominal Volume, it is necessary to start with AVI (abdominal volume index) which calculates the volume of the abdomen in liters, in both sexes and is independent of height, weight, and BMI.

To calculate AVI, it is necessary to measure the abdominal circumference, from the upper edge of the sacrum, and to measure the circumference of the hips.

AVI has been related to impaired glucose tolerance and type II diabetes, but in addition to this, it has been suggested that the concordance of the contextual assessment of Waist circumference and AVI represents a very significant indicator of metabolic syndrome.

$$AVI \text{} formula :$$

$$[ 2 ( Abdomen Circ . ) \times 2 + 0.7 ( Abdomen Circ . - Hip Circ . ) \times 2 ]$$

The Excess Abdominal Volume is obtained by subtracting from the AVI calculated on the patient, the abdominal volume of the same patient at risk 0, or with an abdomen equal to half the height.

Therefore, subtract: [2 (Height/2)×2+0.7 (Height/2−Circ. Hips)×2]

This index is very useful because it monitors the excess abdominal volume in liters, exclusively in patients who have a waist/height ratio>0.5 and who therefore have a risk agreement with the increase in waist circumference.

It is also a very sensitive index that shifts attention from weight to central adiposity and attention to one's body, strongly motivating the patient and representing an excellent index that can also be used in prevention.

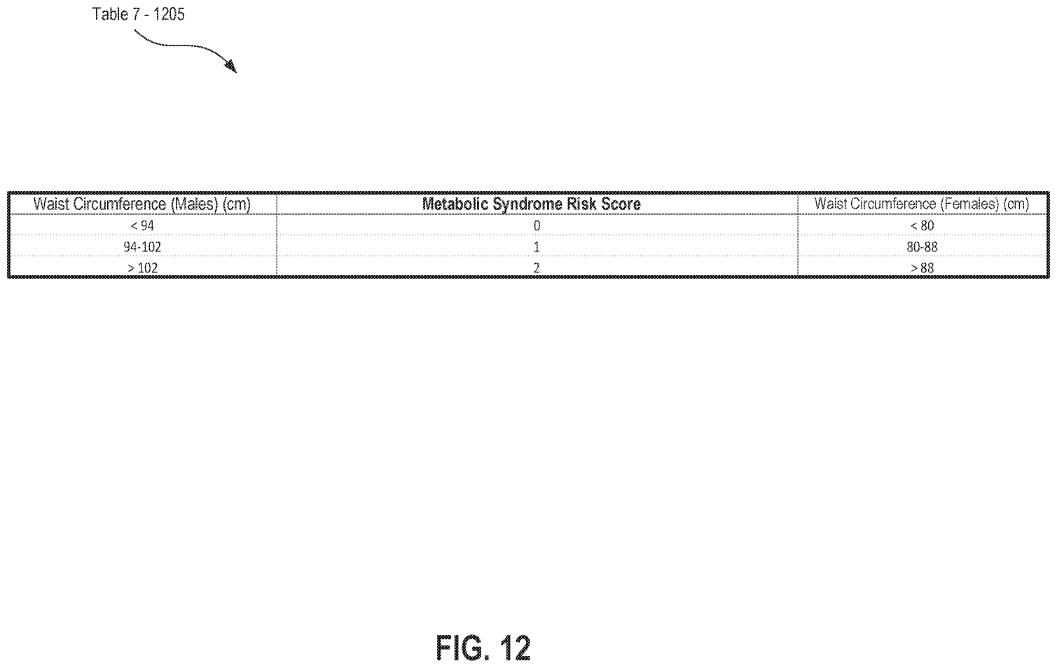

FIG. 12 illustrates an exemplary table (Table 7) determining metabolic syndrome risk score based on waist circumference for both sexes, in accordance with described embodiments. Metabolic syndrome risk score ranges from 0-2 and increases with increased waist circumference for both males and females, with women having lower waist circumference cut-off values for metabolic syndrome risk score.

### Risk Indicators and Related Cut-Offs.

Waist circumference is correlated with cardiometabolic risk and is expressive of central adiposity, but it is also the fulcrum not only of numerous predictive equations, but also of specific relationships with the hips, height, and thigh respectively, whose integrated analysis allows to better define the meaning, risks, and consequences in each person.

The importance of waist circumference is confirmed by studies that show how the measurement of body fat with dual energy X-ray absorptiometry (DXA), which is the gold standard method for body composition, predicts the metabolic syndrome with less precision than the measurement of the waist circumference and to the waist/height ratio.

This consideration greatly diminishes the importance of knowing exactly the percentage of body fat or of attributing a therapeutic goal to its reduction, since the accumulation of fat in the lower body, unlike central obesity, is inversely associated with metabolic risk factors, including hyperinsulinemia, dyslipidemia, and hypertension and is also associated with a reduced incidence of type 2 diabetes mellitus (T2DM) and cardiovascular disease (CVD).

There are, therefore, fundamental differences in the management of lipids that allow the gluteal-femoral fat to promote a metabolic clearance of fats and protect insulin-sensitive tissues from excessive exposure to the same.

It should also be noted that greater overall muscle mass, as well as greater circumference of the pelvis and thigh, are also protective against metabolic risks. Therefore, the use of any surrogate method for the estimation of body fat (plicometry, impedancemetry, etc.), which is known to be prone to measurement errors relative to DXA, has negligible clinical significance. Furthermore, these surrogate methods, compared to direct measurements of circumferences that are clinically significant, also divert the focus from more clinically relevant approaches. Therefore, the overarching principle of the MORPHOGRAM approach is that “measuring is better than estimating.”

For example, it has been calculated that for every centimeter of increase in waist circumference, there is an increase of 66 grams of visceral fat. This data suggests that monitoring of the waist circumference, if practiced habitually, would provide a more significant feedback on health status while motivating and guiding the implementation of appropriate and specific corrective measures. These measures would therefore aim at preventing the progressive increase and metabolic change of the patient towards hyperinsulinism and insulin resistance. This is in contrast to the body weight that one acquires through the scales or more advanced estimates of total body fat, which highlight the problem, but not the problem's origin or how health risk may evolve in the future. As a result, these misleading metrics point to vague and non-specific solutions by addressing the need for reduction of body weight or fat, while ignoring the need for restoring a balanced relationship between food and lifestyle, and how this can positively affect the body.

MORPHOGRAM is the first software that allows an integrated analysis of central fat indicators to draw up a personalized risk profile for each individual. The cut-off values used for waist circumference, Waist/Hip ratio (WHR), Waist/Height ratio (WHtR) are taken from the literature.

The risk factors used for individual risk monitoring include Metabolic Syndrome Risk, Cardiovascular Risk, Adiposity/Muscle WTR Risk (Waist/Thigh Ratio), and NHtR Night Apnea Risk (Neck/Height Ratio).

Metabolic Syndrome Risk simultaneously evaluates in the same individual, the values of the Waist Circumference, the Waist/Hips Ratio and the Waist/Height Ratio (WHtR), whose cut-offs are attributed scores as reported by the international literature.

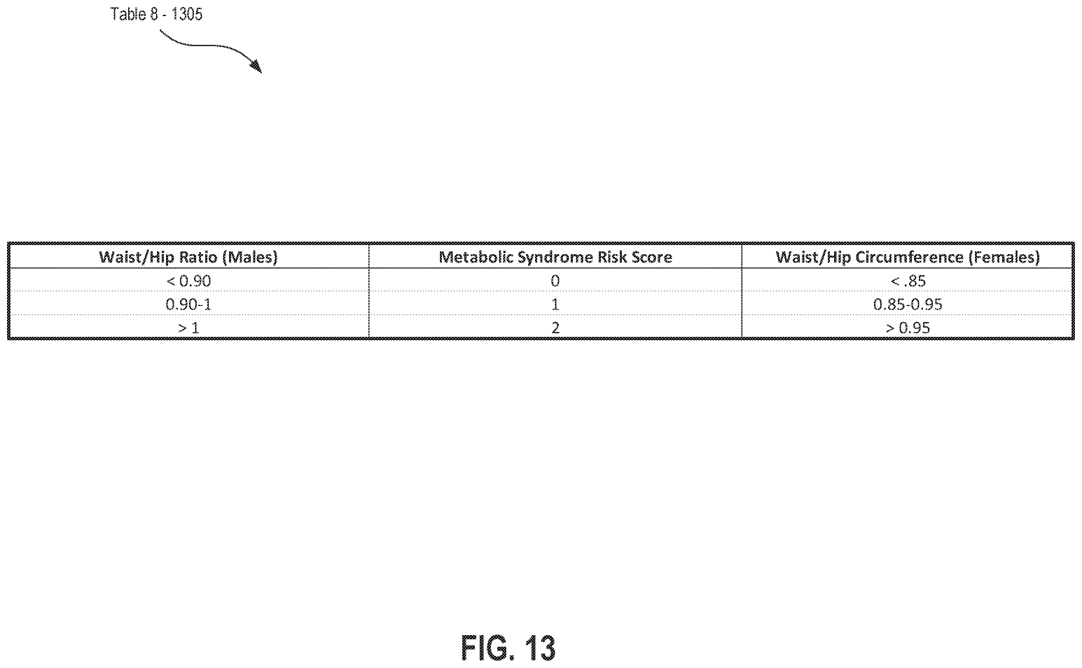

FIG. 13 illustrates an exemplary table (Table 8) determining metabolic syndrome risk score based on waist/hip circumference ratio for both sexes, in accordance with described embodiments.

Metabolic syndrome risk score ranges from 0-2 and increases with increased waist to hip circumference ratio for both males and females, with women having lower waist to hip circumference ratio cut-off values for metabolic syndrome risk score.

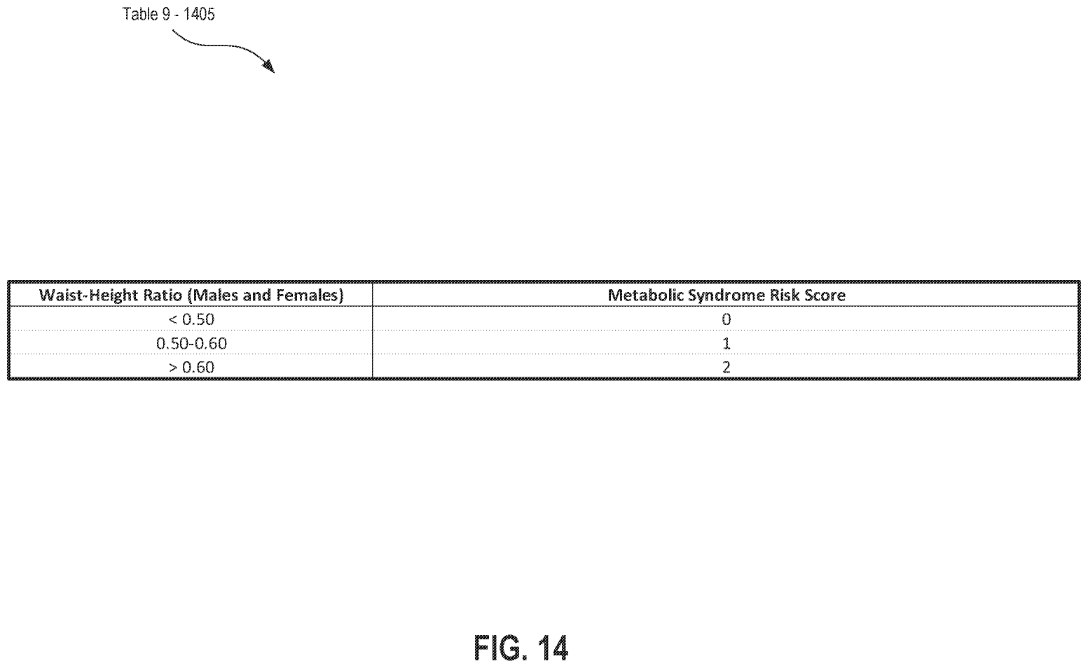

FIG. 14 illustrates an exemplary table (Table 9) determining metabolic syndrome risk score based on waist-height ratio for both sexes, in accordance with described embodiments.

Metabolic syndrome risk score ranges from 0-2 and increases with increased waist-height ratio for both males and females, with women and men having the same waist-height ratio cut-off values for metabolic syndrome risk score.

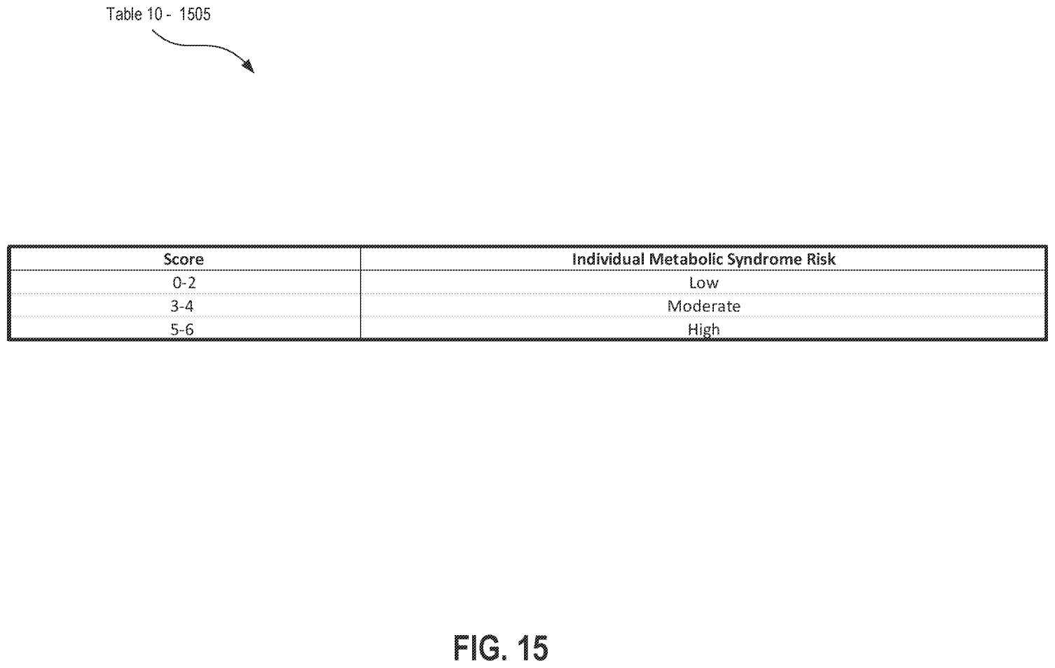

FIG. 15 illustrates an exemplary table (Table 10) classifying individual metabolic syndrome risk based on a risk score, in accordance with described embodiments.

As shown here, individual metabolic syndrome risk score ranges from low (0-2) to high (5-6), with a score of 3-4 considered as being associated with moderate individual metabolic syndrome risk.

Since the risk of waist circumference is inversely correlated with the hips and height, both individual variables, the score is very useful for grading the individual risk of metabolic syndrome, both based on the risk level of each indicator, whether based on the positivity of the indicator or other indicators.

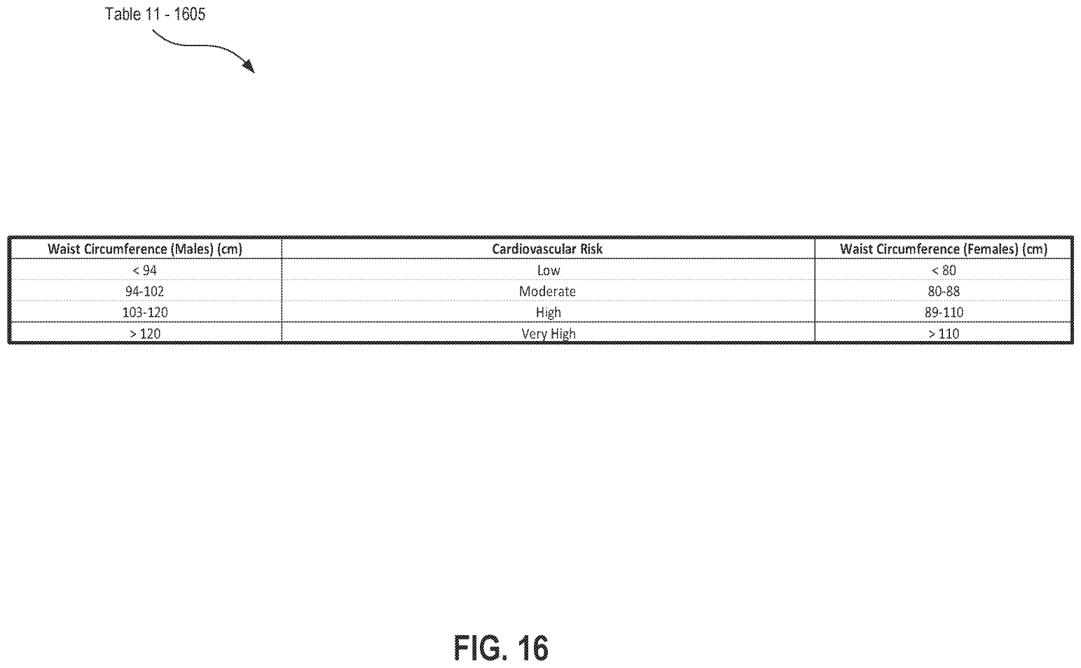

FIG. 16 illustrates an exemplary table (Table 11) classifying cardiovascular risk based on waist circumference for both sexes, in accordance with described embodiments. As shown here, cardiovascular risk includes four risk levels ranges from low to very high, with moderate cardiovascular risk being associated with a waist circumference of 94-102 for men and 80-88 for women. Cardiovascular risk increases with increased waist circumference for both males and females, with women having lower waist circumference cut-off values for cardiovascular risk score.

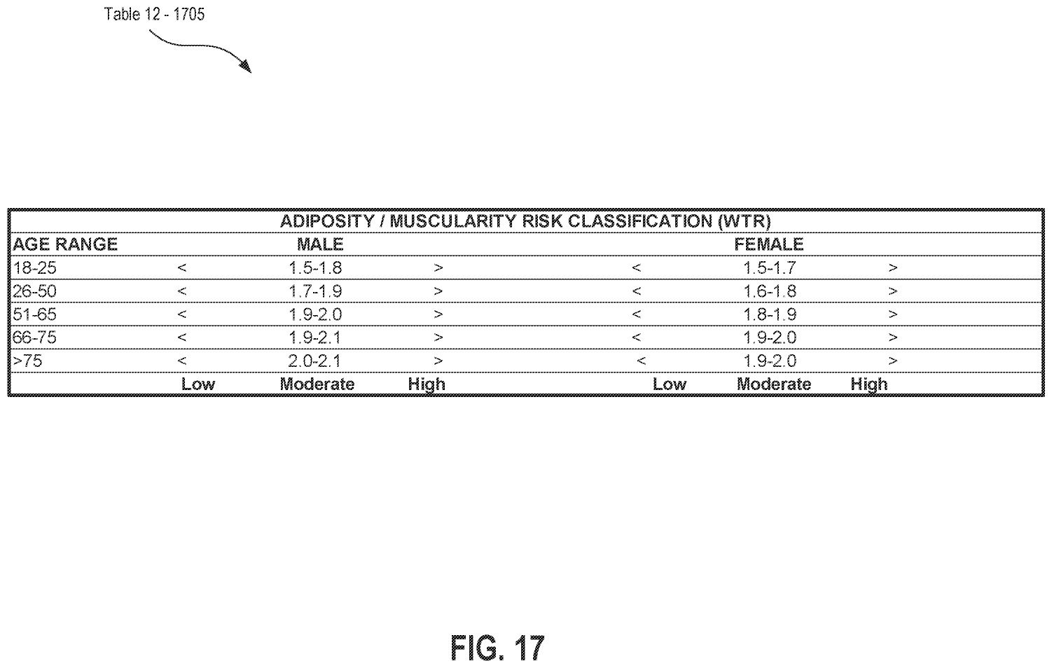

FIG. 17 illustrates an exemplary table (Table 12) providing adiposity/muscularity risk classification based on age and sex, in accordance with described embodiments. Adiposity to muscularity risk classification is based on the ratio of adipose tissue to muscle in a person's body and depends on age and sex. Younger people and women have lower moderate adiposity/muscularity risk ranges.

The Waist/Thigh ratio (WTR) expresses the relationship between the waist circumference, which positively correlates with insulin resistance and diabetes, and the thigh circumference which negatively correlates with them.

The thigh circumference, within the limits of the proportion, with respect to the subject under examination, has a protective function, both when it expresses an increase in lean mass, and when it expresses an increase in subcutaneous fat.

It follows that as the waist circumference increases and the median circumference of the thigh is reduced, the functionality of the lean mass is compromised, glucose tolerance is reduced, and diabetes may appear. Therefore, the increase in the Waist/Thigh ratio, which we have called “Adiposity/Muscularity Risk,” indicates a worsening of the metabolic picture, when the relationship is unbalanced to the advantage of adiposity, as well as the reduction of the same highlights the improvement.

Previous studies found a WTR (waist-to-thigh ratio) for men equal to 1.85+0.2 and 1.63+0.2 for women given subjects of average age. This indicates a sexual dimorphism.

MORPHOGRAM has developed from the respective NHANES (National Health and Nutrition Examination Survey) percentiles for waist and thigh the WTR centiles, which allowed for defining cut-offs by sex and age group and establishing a criterion for the classification of risk.

### NHR Night Apnea Risk (Neck/Height Ratio).

Neck circumference, excluding subjects with thyroid goiter, is correlated with waist circumference and BMI and may be indicative of obesity of the upper half of the body, posing a greater risk of sleep apnea.

The relationship between neck circumference and height (NHR) is an additional risk indicator as it is a predictor of OSA (Obstructive Sleep Apnea) when the value is >0.25.

To give greater sensitivity to this risk indicator, MORPHOGRAM uses it selectively in overweight/obese adult subjects with waist/height ratio>0.5.

### Functional Tests.

The insertion of the data relating to the handgrip functional test is optional but basic to validate the diagnosis of sarcopenia because it associates the observation of muscle loss with the confirmation of the loss of strength.

The finding of values<30 Kg for males and <20 Kg for females can confirm the diagnosis of sarcopenia. However, the possibility of comparing the percentiles of the Lean Mass Indicators with the percentile of the value obtained with the handgrip functional test, can also help to identify subclinical states of debilitation or sarcopenia.

Numerous studies have shown that the handgrip functional test can also be used in the evaluation of nutritional status and that it is a real indicator of the consistency and functionality of lean mass whose percentile is comparable with anthropometric measures.

MORPHOGRAM uses the percentiles of the value in kilograms given by the handgrip functional test across age groups, in order to detect functional decline early and to be able to contrast with a multidimensional treatment approach (nutrition, vitamin D, folic acid, motor reconditioning, rehabilitation, etc.) of the musculoskeletal system and to improve the prognosis of the subject affected by this subtle problem.

Sarcopenia can only be caused by aging or being forced into immobility by disease or disability, but there are promoting conditions such as restrictive eating patterns associated with hypokinetic lifestyles, chronic inflammatory diseases, tumors, and endocrine disorders.

Sarcopenia can also be associated with obesity and is often a consequence of nutritional inadequacy, unbalanced diets, gastrointestinal disorders, malabsorption, etc.

Finally, for a more appropriate classification of sarcopenic states, it is advisable to use the handgrip in association with other tests (such as the chair stand test and walking speed test), while standardizing across such measures.

### Estimation of Total Energy Expenditure (DET).

MORPHOGRAM provides a very important and strategic value to the lean mass (FFM) which is directly proportional to the basal metabolic rate (MB), according to the following equation:

$$MB = FFM \times 19.7 + 413$$

The knowledge of basal metabolism is the pillar on which the estimate of the total energy expenditure (TEE) is based. The basal metabolic rate is a value used both to orient nutritional personalization towards an energy quota consistent with the patient's lifestyle, and to define strategies and nutritional products aimed at achieving the identified objectives.

The total energy expenditure is thus calculated based on the following formula:

$$TEE = BM ( Basal Metabolism ) \times PAL ( Physical Activity Level )$$

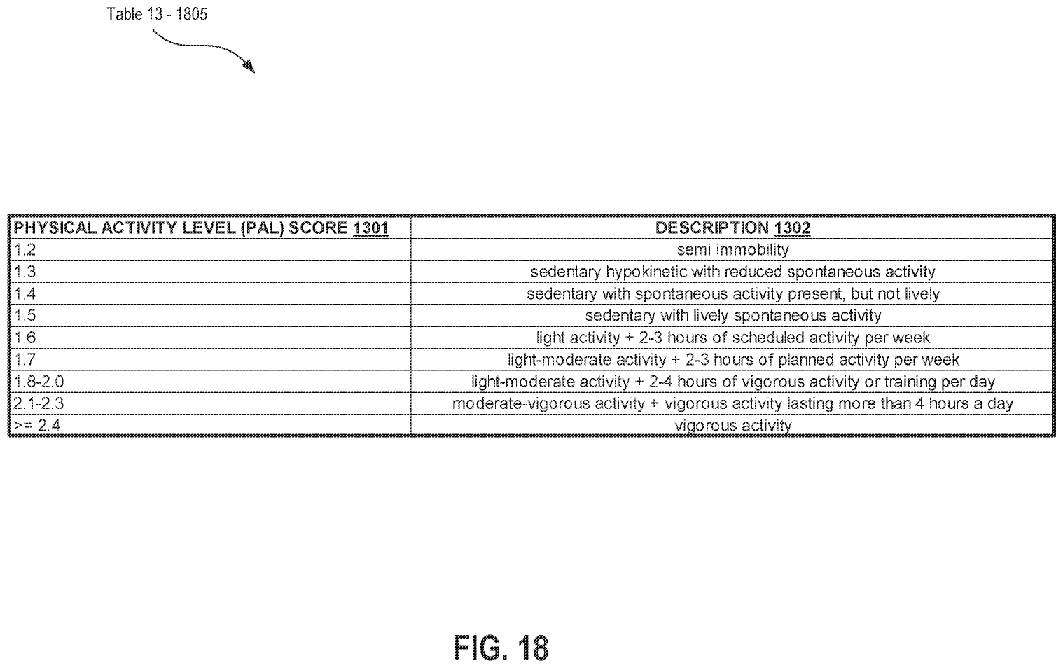

FIG. 18 illustrates an exemplary table (Table 13) describing physical activity level (PAL) based on physical activity level scores, in accordance with described embodiments. PAL scores 1301 range from 1.2 to more than 2.4 and can be described 1302 as semi-immobile for the lowest scores to vigorous activity for the highest scores. Many scores involve a certain number of hours of physical activity per day or week.

PAL is estimated based on the answers provided to a specific question about the usual steps a patient takes in a day, or may be based on the average of the number of steps recorded. The operator of MORPHOGRAM can modulate the PAL, based on a motor history conducted with the patient.

The operator has a drop-down menu that allows him to customize the PAL according to the criteria in Table 13.

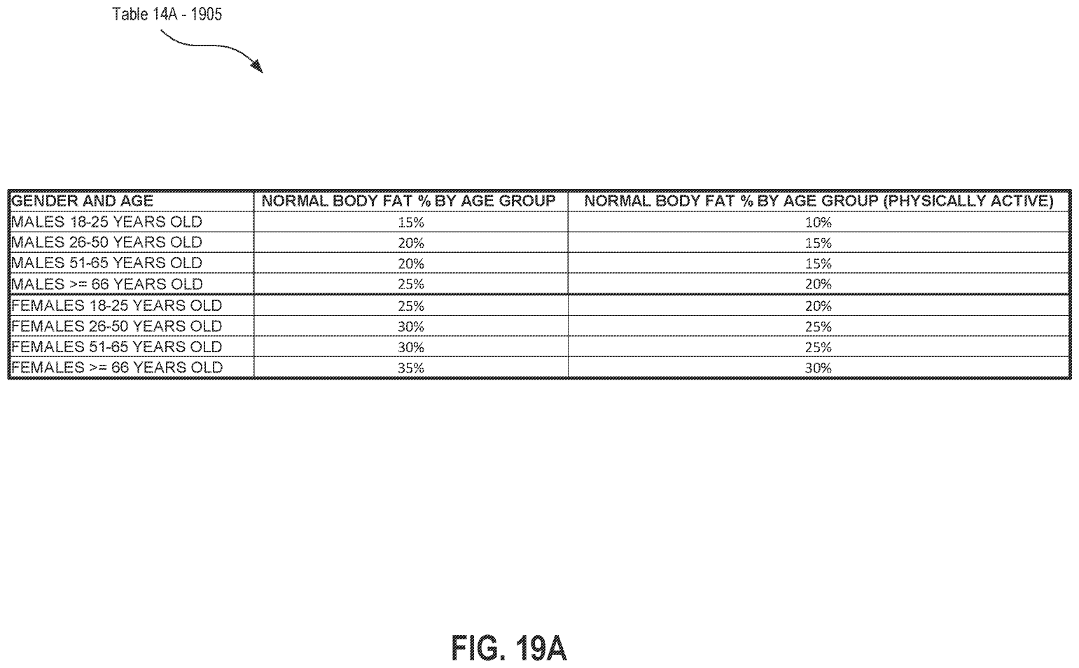

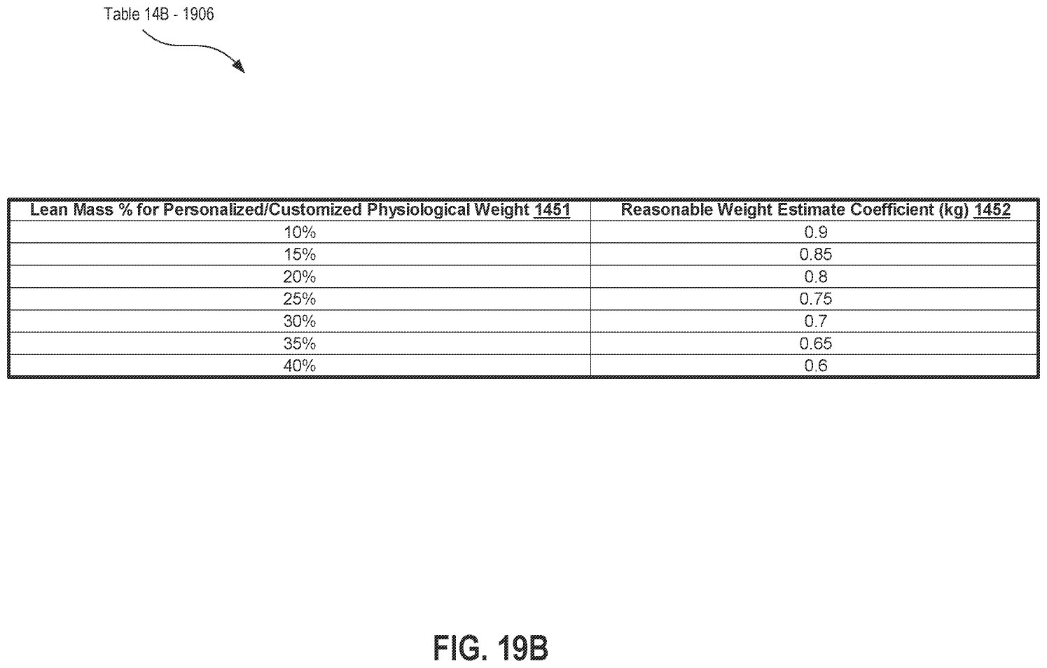

FIG. 19A illustrates an exemplary table (Table 14A) describing normal body fat percentage by age group between both sexes, in accordance with described embodiments. Normal body fat increases with age, with females having higher percentages of normal body fat compared to males, independent of age. For younger age groups in both men and women (i.e., those under age 50), physically active individuals may have a lower normal body fat percentage than those in the same age group who are not physically active.

### Physiological Weight and Reasonable Weight.

Physiological weight is defined as the weight that includes lean mass in balance with the physiological fat percentage (by age group and by sex) and represents the weight that could be achieved after losing excess fat, which often coincides with desired weight.

Since the BMI is nothing more than the sum of the fat free mass index (FFMI) and the fat mass index (FMI), based on the estimates of the latter and their percentiles, MORPHOGRAM evaluates the balances of lean and fat in order to monitor sarcopenic subjects, excluding underweight and subjects with BMI>40.

In the overweight or obese subject, the greatest attention is paid to excess fat and the risk factors of adiposity, but the balance of lean mass to fat mass also plays an important role, both for prognostic purposes and in order to prevent obesity.

In underweight, normal weight, and overweight subjects, attention must be paid to assessing whether the estimated lean mass is adequate with respect to the expected physiological lean mass, based on the constitutional type of the subject in question and the corresponding BMI.

In subjects with morbid obesity (BMI>40), in which the margin of error of the predictive formulas of lean mass increases, the indicators of adiposity and risk will be an indirect measure of the degree of existing sarcopenia that can be monitored.

MORPHOGRAM offers a direct measure of physiological weight, starting from the estimate of the lean mass and using the reference parameters of the fat based on published data.

The physiological weight, calculated in this way, is a good reference in most cases, but it lacks a customization criterion.

FIG. 19B illustrates an exemplary table (Table 14B) describing coefficients for personalized physiological weight based on lean mass estimate, in accordance with described embodiments.

In males, the physiological fat can vary from 10 to 25% and in the female from 20 to 35%, depending on various constitutional, behavioral, and environmental conditions, conditions not foreseen by classification by age group.

It is up to the nutritionist to assess the most appropriate fat percentage for the subject in question, within the ranges indicated for each sex. In practice, nutritionists, if not satisfied with the calculation of the physiological weight based on the percent of the standard physiological fat by age and sex, can customize physiological weight 1451 through a drop-down menu that allows them to obtain the reasonable weight coefficient 1452 based on the percent of fat (lean mass) they deem appropriate for a patient.

The customized physiological weight 1451 is used both to enter the adequate percentage of fat, in various physiological conditions, including sport, and to define the reasonable weight, in order to envisage realistic goals in some particularly obese subjects.

### Constitutional Weight.

To establish physiological weight in underweight, normal weight, and overweight subjects, it is necessary to evaluate the value of the physiological lean mass relative to the normal weight of the constitutional biotype to which a subject belongs to.

MORPHOGRAM, in the context of the normal BMI (BMI between 18.5 and 24.9) attributes ranges of BMI that can be referenced for the respective main constitutional biotypes and their variants:

1.) BMI between 18.5 and 20.5 (slender): long-limbed-ectomorphs, meso-ectomorphs closer to the long-limbed;

2.) BMI between 20.6 and 23.5 (harmonics): leptosomal, normoline-mesomorphic, ectomesomorphic, mesoectomorphic and mesomorphic brevilinear closer to mesomorphs;

3.) BMI between 23.6 and 24.9 (muscular): short mesomorphic and ectomesomorphic with characteristics of robustness; (abdominal): endomorphic short with waist/height ratio>0.5; (subcutaneous) subjects in which the subcutaneous/gynoid component prevails which requires a contextual skin measurement to avoid that the circumferences alone overestimate, in these cases the lean mass.

However, excluding the short endomorph, the endomorphism that appears in the normal weight area highlights the “normal weight obesity”, because in the other cases we will find the BMI above the normal weight levels.

In other words, the MORPHOGRAM algorithm not only defines the constitutional biotypes and an adequate physiological weight, but also indicates the BMI that best represents underweight, normal weight, and overweight subjects.

Once the BMI has been identified, the physiological weight is obtained using the formula: Height 2 (m)×BMI.

By deducting the physiological percentage of body fat by actual patient age group, or the age group deemed most appropriate, the value of the physiological lean mass is obtained.

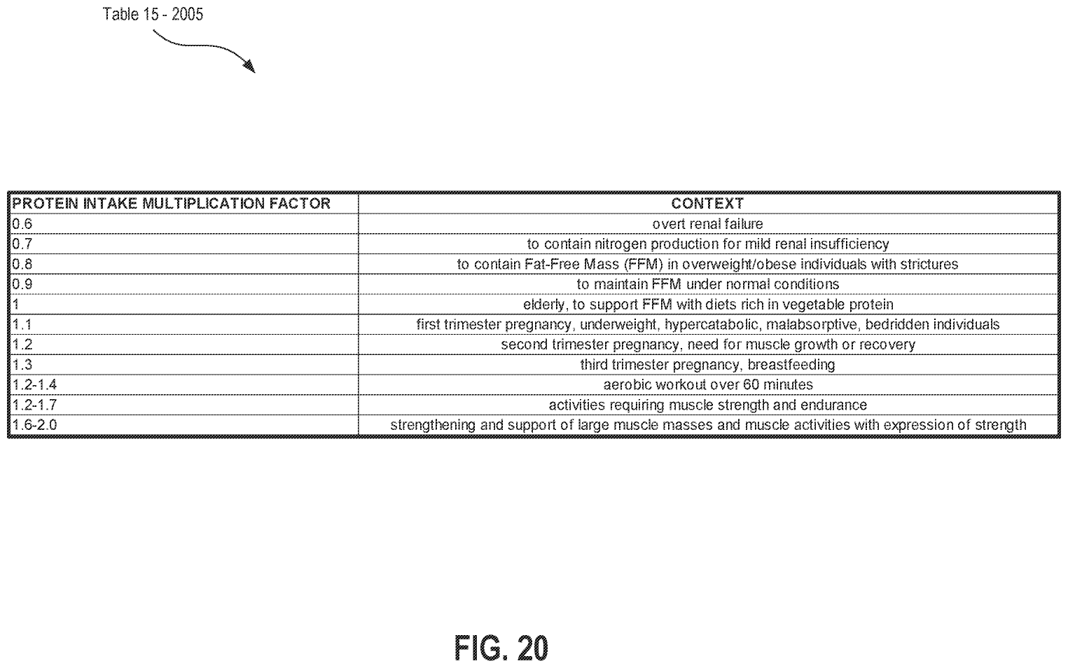

FIG. 20 illustrates an exemplary table (Table 15) describing protein intake multiplication factors and contexts in which to apply them, in accordance with described embodiments.

### Calculation of Protein Needs.

MORPHOGRAM, as we have seen, estimates the value of the lean mass and calculates the physiological weight which contemplates the percentage of physiological fat by age group or a percentage considered appropriate by the nutritionist.

MORPHOGRAM calculates the protein requirement in the measure of 1 g/Kg of physiological weight.

The nutritionist, based on the patient's needs or specific nutritional strategies, can modulate the protein requirement and decide to contain, maintain, recover, or enhance the lean mass, according to identified objectives.

For practical purposes, the nutritionist can customize the protein intake, using a drop-down menu shown in Table 15 which, starting from a physiological weight, allows for the calculation of protein intake requirements.

As shown here, various contexts determine a protein intake multiplier factor to use to account for protein intake needs. Obese individuals and those with kidney diseases have lower protein intake multiplier factors, while physically active individuals and bodybuilders have higher protein intake multiplier factors. A protein intake multiplier factor of 0.9 maintains free-fat mass under normal conditions.

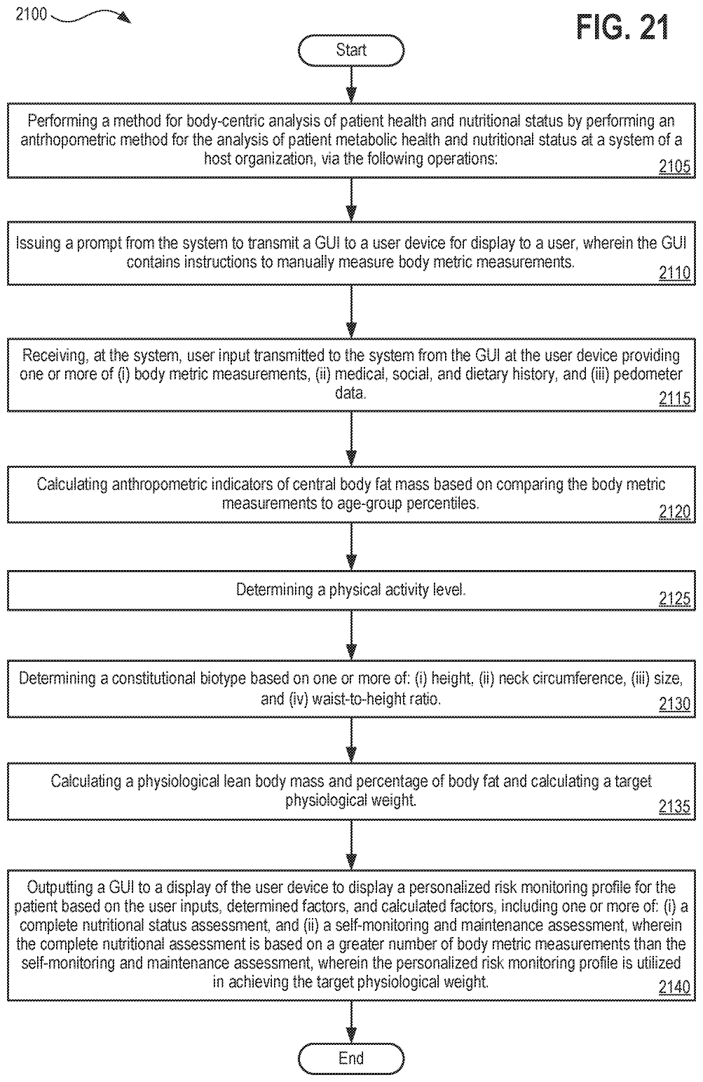

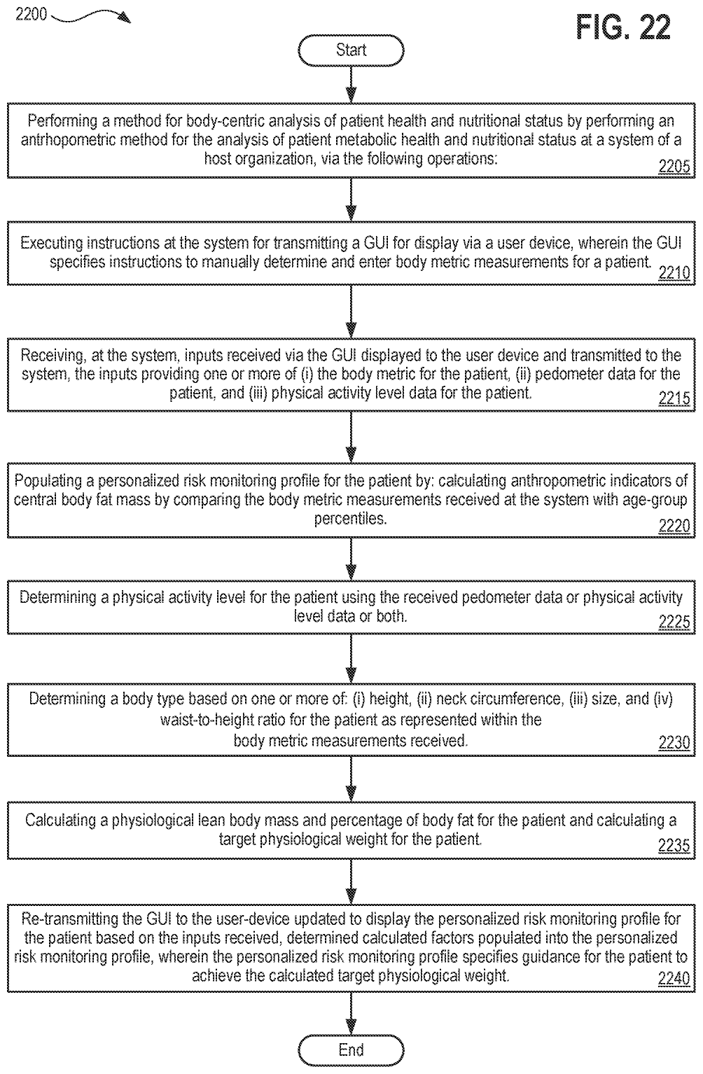

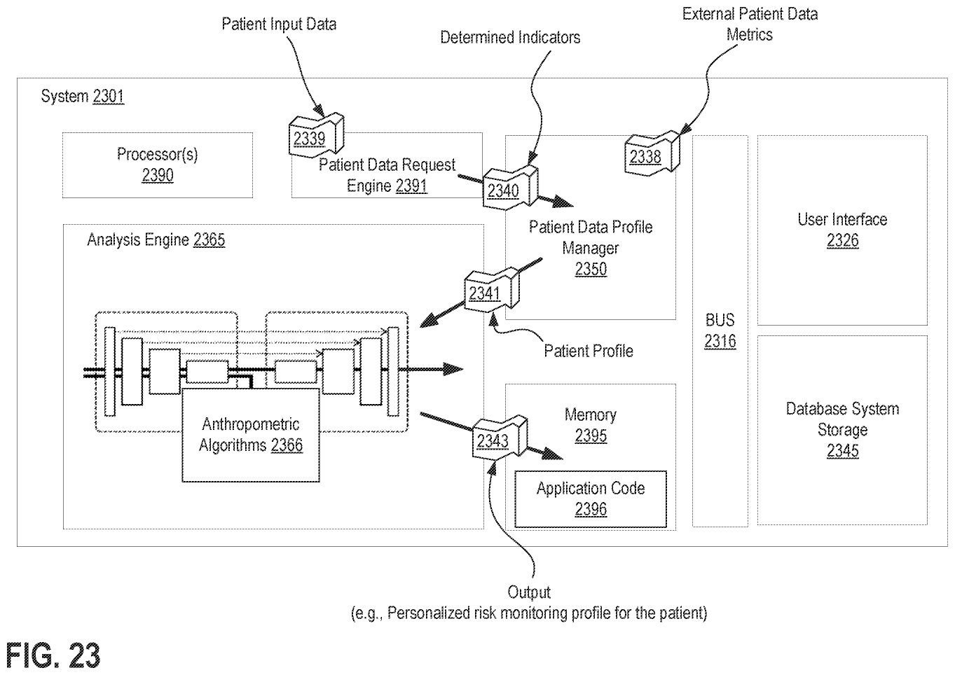

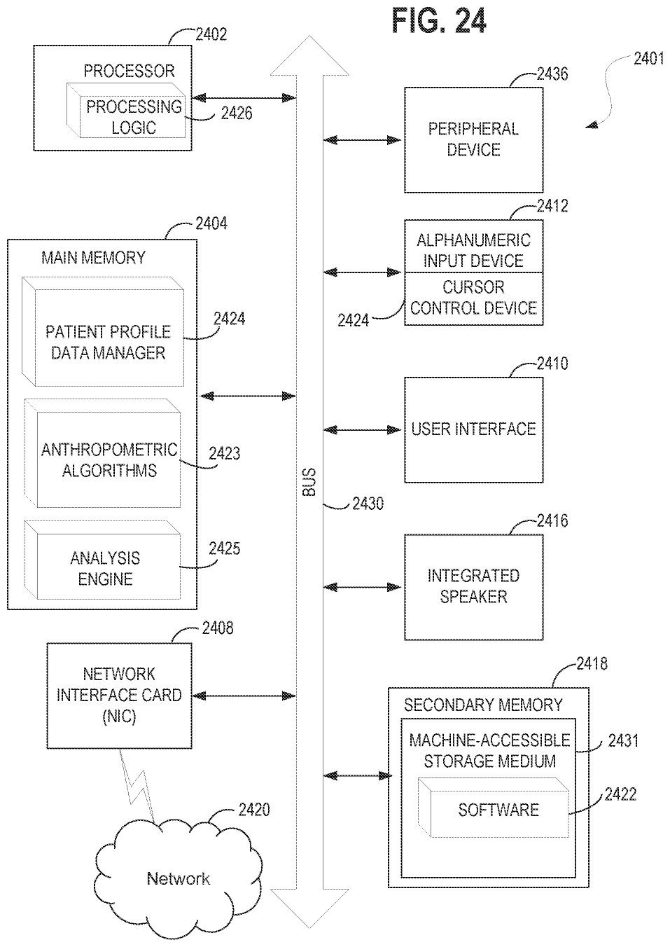

FIGS. 21 and 22 depict flow diagrams illustrating methods 2100 and 2200 for implementing a cloud-based health, nutritional, and body composition analysis platform, in which models are utilized to provide personalized data for health and nutritional counseling, in accordance with disclosed embodiments. Methods 2100 and 2200 may be performed by processing logic that may include hardware (e.g., circuitry, dedicated logic, programmable logic, microcode, etc.), software (e.g., instructions run on a processing device) to perform various operations such as designing, defining, retrieving, parsing, persisting, exposing, loading, executing, operating, receiving, generating, storing, maintaining, creating, returning, presenting, interfacing, communicating, transmitting, querying, processing, providing, determining, triggering, displaying, updating, sending, etc., in pursuance of the systems and methods as described herein. For example, the system 2301 (see FIG. 23) and the machine 2401 (see FIG. 24) and the other supporting systems and components as described herein may implement the described methodologies. Some of the blocks and/or operations listed below are optional in accordance with certain embodiments. The numbering of the blocks presented is for the sake of clarity and is not intended to prescribe an order of operations in which the various blocks must occur.

With reference to the method 2100 depicted at FIG. 21, beginning at block 2105, there is a method performed by a system specially configured for systematically implementing a cloud-based health, nutritional, and body composition analysis platform, in which models are utilized to provide personalized data for health and nutritional counseling by performing an anthropometric method for the analysis of patient metabolic health and nutritional status at a system of a host organization, via the operations set forth at the following blocks. Such a system may be configured with at least a processor and a memory to execute specialized instructions which cause the system to perform the following operations:

At block 2110, processing logic issues a prompt from the system to transmit a GUI to a user device for display to a user, wherein the GUI contains instructions to manually measure body metric measurements.

At block 2115, processing logic receives, at the system, user input transmitted to the system from the GUI at the user device providing one or more of (i) body metric measurements, (ii) medical, social, and dietary history, and (iii) pedometer data.

At block 2120, processing logic calculates anthropometric indicators of central body fat mass based on comparing the body metric measurements to age-group percentiles.

At block 2125, processing logic determines a physical activity level.

At block 2130, processing logic determines a constitutional biotype based on one or more of: (i) height, (ii) neck circumference, (iii) size, and (iv) waist-to-height ratio.

At block 2135, processing logic calculates a physiological lean body mass and percentage of body fat and calculates a target physiological weight.

At block 2140, processing logic outputs a GUI to a display of the user device to display a personalized risk monitoring profile for the patient based on the user inputs, determined factors, and calculated factors, including one or more of: (i) a complete nutritional status assessment, and (ii) a self-monitoring and maintenance assessment, wherein the complete nutritional assessment is based on a greater number of body metric measurements than the self-monitoring and maintenance assessment, wherein the personalized risk monitoring profile is utilized in achieving the target physiological weight.

An alternative variant of the processing methodology is set forth by method 2200 as depicted by FIG. 22. Beginning at block 2205, there is a method performed by a system specially configured for systematically implementing a cloud-based health, nutritional, and body composition analysis platform, in which models are utilized to provide personalized data for health and nutritional counseling by performing an anthropometric method for the analysis of patient metabolic health and nutritional status at a system of a host organization, via the operations set forth at the following blocks.

At block 2210, processing logic executes instructions at the system for transmitting a GUI for display via a user device, wherein the GUI specifies instructions to manually determine and enter body metric measurements for a patient.

At block 2215, processing logic receives, at the system, inputs received via the GUI displayed to the user device and transmitted to the system, the inputs providing one or more of (i) the body metric for the patient, (ii) pedometer data for the patient, and (iii) physical activity level data for the patient.

At block 2220, processing logic populates a personalized risk monitoring profile for the patient by: calculating anthropometric indicators of central body fat mass by comparing the body metric measurements received at the system with age-group percentiles.

At block 2225, processing logic determines a physical activity level for the patient using the received pedometer data or physical activity level data or both.

At block 2230, processing logic determines a body type based on one or more of: (i) height, (ii) neck circumference, (iii) size, and (iv) waist-to-height ratio for the patient as represented within the body metric measurements received.

At block 2235, processing logic calculates a physiological lean body mass and percentage of body fat for the patient and calculating a target physiological weight for the patient.

At block 2240, processing logic re-transmits the GUI to the user-device updated to display the personalized risk monitoring profile for the patient based on the inputs received, determined calculated factors populated into the personalized risk monitoring profile, wherein the personalized risk monitoring profile specifies guidance for the patient to achieve the calculated target physiological weight.

According to another embodiment of method 2100 and 2200, the personalized risk monitoring profile displayed to the user device via the re-transmitted GUI further displays one or more of: a complete nutritional status assessment, and a self-monitoring and maintenance assessment, in which the complete nutritional status assessment is based on a greater number of body metric measurements than the self-monitoring and maintenance.

According to another embodiment of method 2100 and 2200, the physical activity level data for the patient includes one or more of: number of daily steps for the patient; a basal metabolism for the patient; a daily energy expenditure (DET) for the patient; medical data for the patient; social data for the patient; dietary history data for the patient; and activity level assessment data for the patient.

According to another embodiment of method 2100 and 2200, body metric measurements include one or more of: (i) height, (ii) weight, (iii) neck circumference, (iv) mid-arm circumference, (v) forearm circumference, (vi) wrist circumference, (vii) waist circumference, (viii) abdomen circumference, (ix) hip circumference, (x) median thigh circumference, and (xi) pedometer data.

According to another embodiment of method 2100 and 2200, calculating anthropometric indicators based on the body metric measurements includes one or more of: body type indicators, body mass and composition indicators, fat free mass indicators, fat mass indicators, risk factor indicators, and lean mass functionality indicators.

According to another embodiment of method 2100 and 2200, the body type includes one or a combination of two or more of: (i) ectomorph, (ii) leptosomic, (iii) mesomorph, and (iv) endomorph biotypes.

According to another embodiment of method 2100 and 2200, lean body mass is total body weight minus weight due to body fat mass, in which lean body mass is adjusted with an assessment for estimating subcutaneous fat.

According to another embodiment of method 2100 and 2200, the individual risk monitoring profile is based on one or more risk factors includes: (i) metabolic syndrome risk, (ii) cardiovascular risk, (iii) adiposity-muscle waist-thigh risk, and (iv) neck-height ratio night apnea risk.

According to another embodiment of method 2100 and 2200, muscle loss (sarcopenia) is evaluated via a handgrip functional test.

According to another embodiment of method 2100 and 2200, patient protein intake is customized to modify the physiological lean body mass.

According to another embodiment of method 2100 and 2200, the physiological lean mass estimates a lean mass deficit.

According to another embodiment of method 2100 and 2200, the target physiological weight represents a weight after losing excess fat to balance physiological lean body mass with physiological fat percentage for a patient.

According to another embodiment of method 2100 and 2200, either the patient or a clinician authenticates at the user device and receives the GUI transmitted from the system at the user-device displaying the personalized risk monitoring profile for the patient.

According to a particular embodiment, there is a non-transitory computer-readable storage medium having instructions stored thereupon that, when executed by a system having at least a processor and a memory therein, the instructions cause the processor to perform operations including: executing instructions at the system for transmitting a GUI for display via a user device, wherein the GUI specifies instructions to manually determine and enter body metric measurements for a patient; receiving, at the system, inputs received via the GUI displayed to the user device and transmitted to the system, the inputs providing one or more of (i) the body metric for the patient, (ii) pedometer data for the patient, and (iii) physical activity level data for the patient; populating a personalized risk monitoring profile for the patient by: calculating anthropometric indicators of central body fat mass by comparing the body metric measurements received at the system with age-group percentiles; determining a physical activity level for the patient using the received pedometer data or physical activity level data or both; determining a body type based on one or more of: (i) height, (ii) neck circumference, (iii) size, and (iv) waist-to-height ratio for the patient as represented within the body metric measurements received; calculating a physiological lean body mass and percentage of body fat for the patient; calculating a target physiological weight for the patient; and re-transmitting the GUI to the user-device updated to display the personalized risk monitoring profile for the patient based on the inputs received, determined calculated factors populated into the personalized risk monitoring profile, wherein the personalized risk monitoring profile specifies guidance for the patient to achieve the calculated target physiological weight.

FIG. 23 shows a diagrammatic representation of a system 2301 within which embodiments may operate, be installed, integrated, or configured. In accordance with one embodiment, there is a system 2301 having at least a processor 2390 and a memory 2395 therein to execute implementing application code 2396. Such a system 2301 may communicatively interface with and cooperatively execute with the benefit of remote systems, such as a user device sending instructions and data, a user device to receive as an output 2343 from the system 2301 the personalized risk monitoring profile for the patient (e.g., output 2343) having been processed in accordance with the anthropometric algorithms 2366 via the analysis engine 2365. Further depicted is the receiving of patient input data 2339 via the patient data request engine 2391 as well as the determined indicators 2340 for a patient which are provided to the patient data profile manager 2350. The patient data profile manager 2350 may additionally receive and consume external patient data metrics 2338, such as social media data, estimated fitness activity data, historical health and fitness data, or data shared by other apps, such as apps for tracking running, walking, biking, weightlifting, and so forth. Ultimately, the patient profile 2341 is transmitted from the patient data profile manager 2350 to the analysis engine 2365 which performs the anthropometric algorithms 2366 to generate and yield from the system the output 2343 which includes the personalized risk monitoring profile for the patient.

According to the depicted embodiment, the system 2301, includes a processor 2390 and the memory 2395 to execute instructions at the system 2301. The system 2301 as depicted here is specifically customized and specially configured to systematically implement a cloud-based health, nutritional, and body composition analysis platform, in which models are utilized to provide personalized data for health and nutritional counseling by performing an anthropometric method for the analysis of patient metabolic health and nutritional status at a system of a host organization. As shown here, the system is specially configured to execute the instructions stored in the memory via the processor to cause the system to perform operations including: executing instructions at the system 2301 for transmitting a GUI for display via a user device (e.g., such as transmitting the GUI to the user device via the user interface 2326), wherein the GUI specifies instructions to manually determine and enter body metric measurements for a patient; receiving, at the system, inputs (2390) received via the GUI displayed to the user device and transmitted to the system 2301, the inputs providing one or more of (i) the body metric for the patient, (ii) pedometer data for the patient, and (iii) physical activity level data for the patient (e.g., via one or more of the patient input data 2339, the determined indicators 2340, or the external patient data metrics 2338); populating a personalized risk monitoring profile (e.g., patient profile 2341) for the patient by: calculating anthropometric indicators of central body fat mass by comparing the body metric measurements received at the system with age-group percentiles (e.g., via anthropometric algorithms 2366); determining a physical activity level for the patient using the received pedometer data or physical activity level data or both; determining a body type based on one or more of: (i) height, (ii) neck circumference, (iii) size, and (iv) waist-to-height ratio for the patient as represented within the body metric measurements received; calculating a physiological lean body mass and percentage of body fat for the patient; calculating a target physiological weight for the patient; and re-transmitting the GUI to the user-device updated to display the personalized risk monitoring profile (e.g., provided as output 2343) for the patient based on the inputs received, determined calculated factors populated into the personalized risk monitoring profile, wherein the personalized risk monitoring profile specifies guidance for the patient to achieve the calculated target physiological weight (e.g., with the guidance generated via the analysis engine 2365).

According to another embodiment of the system 2301, a user interface 2311 communicably interfaces with a user client device remote from the system and communicatively interfaces with the system via a public Internet.

Bus 2311 interfaces the various components of the system 2301 amongst each other, with any other peripheral(s) of the system 2301, and with external components such as external network elements, other machines, client devices, cloud computing services, etc. Communications may further include communicating with external devices via a network interface over a LAN, WAN, or the public Internet.

FIG. 24 illustrates a diagrammatic representation of a machine 2401 in the exemplary form of a computer system, in accordance with one embodiment, within which a set of instructions, for causing the machine/computer system to perform any one or more of the methodologies discussed herein, may be executed.

In alternative embodiments, the machine may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 2401 includes a processor 2402, a main memory 2404 (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory 2418 (e.g., a persistent storage device including hard disk drives and a persistent database and/or a multi-tenant database implementation), which communicate with each other via a bus 2430. Main memory 2404 includes instructions for executing a patient profile data manager 2424 and the anthropometric algorithms 2423 having been specially configured for use by the analysis engine 2425 which applies the methodologies for systematically implementing a cloud-based health, nutritional, and body composition analysis platform, in which models are utilized to provide personalized data for health and nutritional counseling by performing an anthropometric method for the analysis of patient metabolic health and nutritional status at a system of a host organization, as described herein and in support of the methodologies discussed herein.

Processor 2402 represents one or more specialized and specifically configured processing devices such as a microprocessor, central processing unit, or the like. More particularly, the processor 2402 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 2402 may also be one or more special-purpose processing devices such as an application-specific integrated circuit (ASIC), a field programmable gate array (FPGA), a digital signal processor (DSP), network processor, or the like. Processor 2402 is configured to execute the processing logic 2411 for performing the operations and functionality which is discussed herein.

The computer system 2401 may further include a network interface card 2408. The computer system 2401 also may include a user interface 2410 (such as a video display unit, a liquid crystal display, etc.), an alphanumeric input device 2412 (e.g., a keyboard), a cursor control device 2418 (e.g., a mouse), and a signal generation device 2411 (e.g., an integrated speaker). The computer system 2401 may further include peripheral device 2436 (e.g., wireless or wired communication devices, memory devices, storage devices, audio processing devices, video processing devices, etc.).

The secondary memory 2418 may include a non-transitory machine-readable storage medium or a non-transitory computer readable storage medium or a non-transitory machine-accessible storage medium 2431 on which is stored one or more sets of instructions (e.g., software 2422) embodying any one or more of the methodologies or functions described herein. The software 2422 may also reside, completely or at least partially, within the main memory 2404 and/or within the processor 2402 during execution thereof by the computer system 2401, the main memory 2404 and the processor 2402 also constituting machine-readable storage media. The software 2422 may further be transmitted or received over a network 2420 via the network interface card 2408.

It is therefore described herein, systems, methods, and apparatuses which are specially configured to execute and to expose as a cloud computing MORPHOGRAM platform, the necessary systems, methods, and apparatuses for performing an anthropometric method for the analysis of patient metabolic health and nutritional status.

Such capabilities are not currently met by the marketplace nor are they known to those familiar with the relevant technical arts. Specifically, there is no prior cloud-platform that accepts the body measurements, fitness, and lifestyle, and then returns a personalized risk monitoring profile for the patient in the manner provided by the Morphogram platform. The described methodologies and specially configured systems are unique compared with all others because they are based on the principle that a wider range of information is provided to the system to assist health professionals in making data-driven decisions and thus, enabling such health professionals to provide personalized recommendations to their patients. In fact, devices previously utilized for assessing patients in clinical nutrition practice are hardware tools that analyze only body composition in terms of the relationship between lean mass and fat mass. Moreover, the majority of cloud services available to the marketplace are merely diet planners and CRMs. While a subset does allow for the collection of body measurements including body circumferences and skin folds, such platforms provide only standard information related to international cut-offs.

In contrast, the Morphogram platform and unique methodology brings important improvements to the assessment of body composition and metabolic health risks evaluation because: (1) The Morphogram platform implements a new bespoke algorithm as is described above for the estimation of fat mass and lean mass which is reliable and not set on a specific population but rather, on the variation of central adiposity which represents more than 90% of the entire body fat variation; (2) the Morphogram platform utilizes a new bespoke algorithm as described above for the assessment of metabolic syndrome risk in which the platform provides, for each patient, their personal risk of metabolic syndrome without requiring the use of biomarkers; (3) the Morphogram platform utilizes a new bespoke algorithm as described above for the evaluation of the abdominal fat and further, as described above, the Morphogram platform has creates and utilizes a new indicator (specifically the Abdominal Volume Excess) that enables the monitoring of central adiposity and its relation with health risks, thus going beyond weight-loss and BMI metrics as were utilized previously.

Furthermore, the Morphogram platform is the first such platform and methodology to utilize the waist-to-thigh ratio (WTR) not just for assessing the diabetes risk of a patient, but also for assessing sarcopenic risk, recognizing the fact that people with diabetes tend to lose muscle mass in the gluteal-femoral area which is shown through research to be directly connected with key body functionalities, such as walking and standing up from a chair. Similarly, the Morphogram platform is the first to use the neck-to-height ratio (NHR) to assess the sleep apnea risk of a patient being assessed.

Simply stated, the Morphogram platform notably implements a new and unique algorithm to evaluate the Body Type of a patient in relation to metabolic health risks.

In so doing, the Morphogram platform and related methodologies as described herein provide the following technical solution, as provided by the cloud based Morphogram platform, beyond the mere applications of the described algorithms, thus overcoming technical obstacles historically in place which prevented others from taking the body measurements and returning a personalized risk monitoring profile in the manner accomplished by the Morphogram platform described herein. Notably, prior techniques for assessing body composition were mostly accomplished via the use of with hardware tools, such as bioelectrical impedance and plicometry. Unfortunately, these prior known solutions do not allow for the remote monitoring of patients because the use of bioelectrical impedance requires physical patient presence, whereas plicometry requires technical expertise when measuring the skin folds of the patient.

By implementing the technical solution in the manner described, the Morphogram platform makes the remote monitoring of a patient's body composition and metabolic health risks an easy-to-practice reality for patients which lack the traditionally required technical knowledge as well as eliminates the need for on-site and in-person physical accessibility to the patient by a healthcare professional. This is because the Morphogram platform is specially configured to implement two types of complementary assessments, specifically, (1) a full assessment utilizing 8 body circumferences of the patient, which is preferably captured during a first appointment and secondly (2) the smart assessment which captures and utilizes 3 body circumferences which are preferably captured during follow-up appointments and which are utilized by the Morphogram platform to reduce the potential for error by patients when self-monitoring during online consultations.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A system comprising:
a memory to store instructions;
a processor to execute the instructions stored in the memory;
wherein the system is specially configured to execute the instructions stored in the memory via the processor to cause the system to perform operations including:
executing instructions at the system for transmitting a GUI for display via a user device, wherein the GUI specifies instructions to manually determine and enter body metric measurements for a patient;
receiving, at the system, inputs received via the GUI displayed to the user device and transmitted to the system, the inputs providing one or more of (i) the body metric for the patient, (ii) pedometer data for the patient, and (iii) physical activity level data for the patient;
populating a personalized risk monitoring profile for the patient by:
calculating anthropometric indicators of central body fat mass by comparing the body metric measurements received at the system with age-group percentiles;
determining a physical activity level for the patient using the received pedometer data or physical activity level data or both;
determining a body type based on one or more of: (i) height, (ii) neck circumference, (iii) size, and (iv) waist-to-height ratio for the patient as represented within the body metric measurements received;
calculating a physiological lean body mass and percentage of body fat for the patient;
calculating a target physiological weight for the patient; and
re-transmitting the GUI to the user-device updated to display the personalized risk monitoring profile for the patient based on the inputs received, determined calculated factors populated into the personalized risk monitoring profile, wherein the personalized risk monitoring profile specifies guidance for the patient to achieve the calculated target physiological weight.

2. The system of claim 1, wherein the personalized risk monitoring profile displayed to the user device via the re-transmitted GUI further displays one or more of:
a complete nutritional status assessment, and
a self-monitoring and maintenance assessment, wherein the complete nutritional status assessment is based on a greater number of body metric measurements than the self-monitoring and maintenance.

3. The system of claim 1, wherein the physical activity level data for the patient includes one or more of:
number of daily steps for the patient;
a basal metabolism for the patient;
a daily energy expenditure (DET) for the patient;
medical data for the patient;
social data for the patient;
dietary history data for the patient; and
activity level assessment data for the patient.

4. The system of claim 1, wherein body metric measurements include one or more of: (i) height, (ii) weight, (iii) neck circumference, (iv) mid-arm circumference, (v) forearm circumference, (vi) wrist circumference, (vii) waist circumference, (viii) abdomen circumference, (ix) hip circumference, (x) median thigh circumference, and (xi) pedometer data.

5. The system of claim 1, wherein calculating anthropometric indicators based on the body metric measurements includes one or more of: body type indicators, body mass and composition indicators, fat free mass indicators, fat mass indicators, risk factor indicators, and lean mass functionality indicators.

6. The system of claim 1, wherein the body type includes one or a combination of two or more of: (i) ectomorph, (ii) leptosomic, (iii) mesomorph, and (iv) endomorph biotypes.

7. The system of claim 1, wherein lean body mass is total body weight minus weight due to body fat mass, wherein lean body mass is adjusted with an assessment for estimating subcutaneous fat.

8. The system of claim 1, wherein the individual risk monitoring profile is based on one or more risk factors includes: (i) metabolic syndrome risk, (ii) cardiovascular risk, (iii) adiposity-muscle waist-thigh risk, and (iv) neck-height ratio night apnea risk.

9. The system of claim 1, wherein muscle loss (sarcopenia) is evaluated via a handgrip functional test.

10. The system of claim 1, wherein patient protein intake is customized to modify the physiological lean body mass.

11. The system of claim 1, wherein the physiological lean mass estimates a lean mass deficit.

12. The system of claim 1, wherein the target physiological weight represents a weight after losing excess fat to balance physiological lean body mass with physiological fat percentage for a patient.

13. The system of claim 1, wherein either the patient or a clinician authenticates at the user device and receives the GUI transmitted from the system at the user-device displaying the personalized risk monitoring profile for the patient.

14. A method for body-centric analysis of patient health and nutritional status performed by a system of a host organization having at least a processor and a memory therein to execute instructions, wherein the method comprises:
executing instructions at the system for transmitting a GUI for display via a user device, wherein the GUI specifies instructions to manually determine and enter body metric measurements for a patient;
receiving, at the system, inputs received via the GUI displayed to the user device and transmitted to the system, the inputs providing one or more of (i) the body metric for the patient, (ii) pedometer data for the patient, and (iii) physical activity level data for the patient;
populating a personalized risk monitoring profile for the patient by:
calculating anthropometric indicators of central body fat mass by comparing the body metric measurements received at the system with age-group percentiles;
determining a physical activity level for the patient using the received pedometer data or physical activity level data or both;
determining a body type based on one or more of: (i) height, (ii) neck circumference, (iii) size, and (iv) waist-to-height ratio for the patient as represented within the body metric measurements received;
calculating a physiological lean body mass and percentage of body fat for the patient;
calculating a target physiological weight for the patient; and
re-transmitting the GUI to the user-device updated to display the personalized risk monitoring profile for the patient based on the inputs received, determined calculated factors populated into the personalized risk monitoring profile, wherein the personalized risk monitoring profile specifies guidance for the patient to achieve the calculated target physiological weight.

15. The method of claim 14, wherein body metric measurements include one or more of: (i) height, (ii) weight, (iii) neck circumference, (iv) mid-arm circumference, (v) forearm circumference, (vi) wrist circumference, (vii) waist circumference, (viii) abdomen circumference, (ix) hip circumference, (x) median thigh circumference, and (xi) pedometer data.

16. The method of claim 14, wherein calculating anthropometric indicators based on the body metric measurements includes one or more of: body type indicators, body mass and composition indicators, fat free mass indicators, fat mass indicators, risk factor indicators, and lean mass functionality indicators.

17. The method of claim 14, wherein the body type includes one or a combination of two or more of: (i) ectomorph, (ii) leptosomic, (iii) mesomorph, and (iv) endomorph biotypes.

18. Non-transitory computer readable storage media having instructions stored thereupon that, when executed by a system having at least a processor and a memory therein, the instructions cause the system to perform operations including:
executing instructions at the system for transmitting a GUI for display via a user device, wherein the GUI specifies instructions to manually determine and enter body metric measurements for a patient;
receiving, at the system, inputs received via the GUI displayed to the user device and transmitted to the system, the inputs providing one or more of (i) the body metric for the patient, (ii) pedometer data for the patient, and (iii) physical activity level data for the patient;
populating a personalized risk monitoring profile for the patient by:
calculating anthropometric indicators of central body fat mass by comparing the body metric measurements received at the system with age-group percentiles;
determining a physical activity level for the patient using the received pedometer data or physical activity level data or both;
determining a body type based on one or more of: (i) height, (ii) neck circumference, (iii) size, and (iv) waist-to-height ratio for the patient as represented within the body metric measurements received;
calculating a physiological lean body mass and percentage of body fat for the patient;
calculating a target physiological weight for the patient; and
re-transmitting the GUI to the user-device updated to display the personalized risk monitoring profile for the patient based on the inputs received, determined calculated factors populated into the personalized risk monitoring profile, wherein the personalized risk monitoring profile specifies guidance for the patient to achieve the calculated target physiological weight.

19. The non-transitory computer readable storage media of claim 18, wherein body metric measurements include one or more of: (i) height, (ii) weight, (iii) neck circumference, (iv) mid-arm circumference, (v) forearm circumference, (vi) wrist circumference, (vii) waist circumference, (viii) abdomen circumference, (ix) hip circumference, (x) median thigh circumference, and (xi) pedometer data.

20. The non-transitory computer readable storage media of claim 18, wherein calculating anthropometric indicators based on the body metric measurements includes one or more of: body type indicators, body mass and composition indicators, fat free mass indicators, fat mass indicators, risk factor indicators, and lean mass functionality indicators.

21. The non-transitory computer readable storage media of claim 18, wherein the body type includes one or a combination of two or more of: (i) ectomorph, (ii) leptosomic, (iii) mesomorph, and (iv) endomorph biotypes.
